(19) United States
(12) Patent Application Publication
(10) Pub. No.: US 2025/0249064 A1
(43) Pub. Date: Aug. 7, 2025

(54) COMPOSITION FROM ARONIA MELANOCARPA

(71) Applicant: GIVAUDAN SA, VERNIER (CH)

(72) Inventors: Manon LECOMTE, Avignon (FR); Pascale Elizabeth Renée FANÇA-BERTHON, Avignon (FR); Emilie Annie Claudie FROMENTIN, South Hackensack, NJ (US); Melanie Marie-Lou LE SAYEC, London (GB); Ana Maria RODRIGUEZ MATEOS, Guildford (GB)

(21) Appl. No.: 18/705,256

(22) PCT Filed: Oct. 14, 2022
(86) PCT No.: PCT/EP2022/078598

§ 371 Date: Apr. 26, 2024

Related U.S. Application Data
(60) Provisional application No. 63/273,635, filed on Oct. 29, 2021.

(51) Int. Cl. A61K 36/73; A61P 9/12; C12Q 1/02; G01N 33/569

(52) U.S. Cl. CPC A61K 36/73; A61P 9/12; C12Q 1/025; G01N 33/56911; A61K 2236/33

(57) ABSTRACT

The present invention relates to compositions for use in treating cardiovascular disease (CVD) and/or gut dysbiosis, and methods of treating CVD and/or gut dysbiosis. In particular, the present invention relates to compositions for use and methods for treating cardiovascular disease (CVD) and/or gut dysbiosis in subject groups having a reduced gut gene count (faecal microbiome gene count).

### FIELD OF THE INVENTION

The present invention relates to compositions for use in treating and/or preventing cardiovascular disease (CVD) and/or gut dysbiosis, and methods of treating CVD and/or gut dysbiosis. In particular, the present invention relates to compositions for use and methods for treating cardiovascular disease (CVD) and/or gut dysbiosis in subject groups having a reduced gut gene count (faecal microbiome gene count).

### BACKGROUND OF THE INVENTION

CVD, also called heart diseases, is an umbrella term used to refer to conditions affecting the heart or circulation, and includes coronary artery disease, stroke, and hypertension. CVDs are the first cause of mortality worldwide, responsible for an estimated 31% of all deaths globally (World Health Organisation 2017). Each year, CVD accounts for almost 27% of the deaths in the UK and the whole healthcare cost related to CVD in the UK is estimated at £9 billion per year (British Heart Foundation).

Atherosclerosis refers to the progressive disease characterized by the build-up of lipids and fibrous plaques in arteries, leading to hardening and narrowing of these large vessels. Atherosclerosis occurs in the most internal of the 3 layers of the artery: the tunica intima.

Cardiovascular events can occur due to an accumulation of atherosclerotic plaques obstructing or narrowing the arterial lumen. Atherosclerosis-based CVD is one of the main causes of vascular disease worldwide. Indeed, after a slow progression during lifetime, the disease can eventually lead to peripheral vascular diseases and/or stroke among the older general population. Some risk factors have been related to the onset and development of atherosclerosis, such as hypercholesterolemia, hypertension, cigarette smoking and diabetes mellitus.

Blood pressure (BP) has been shown to be a robust surrogate marker of CVD risk and a decrease of 3 mmHg in systolic BP (SBP) was associated with a decrease in the risk of CVD mortality by 5%.

Although mechanisms behind the link between BP and atherosclerosis have not been fully identified yet, the importance of the endothelium and the process of oxidative stress occurring at its level seem to be the main explanation. However, while NO exerts a key role in the regulation of vascular tone and BP, it has also been observed that compromised NO activity is an essential element of the onset of hypertension. The pulsatile factor of BP has been shown to induce atheroma instability and eventually, plaque rupture, indicating the mediating role of BP on atherosclerosis.

The build-up of atherosclerotic plaques leads to stiffening of the arterial walls.

Pulse wave velocity (PWV) is the most used technique to assess arterial stiffness. PWV uses applanation tonometry technology to measure the pressure waves speed traveling in the arterial system. PWV, expressed in m/s, is calculated based on the division of the distance between the two points of measurement by the pulse pressure wave transit time at these two locations. In common practice, carotid and femoral artery are the preferred sites for the assessment of PWV, which is then designated as Cf-PWV.

Augmentation index (AIx) assessment is another useful tool for the measurement of arterial stiffness which has been developed over the last decades. Using the same applanation tonometry technique as for PWV, AIx is non-invasive method estimating the pulse wave reflections of the arteries which is expressed as a percentage. PWV and AIx represents two different measures of arterial stiffness which are not interchangeable.

The mechanisms behind AIx estimation and its relation to CVD and atherosclerosis have not been fully elucidated yet. AIx has been shown to increase significantly with age, following a curvilinear pattern. However, it has been suggested that the increase in AIx could be related to an elevation of the ROS level, which constitutes one of the early stages of the development of atherosclerosis. Indeed, a study showed AIx was associated to serum ROS concentration in a population of smokers. This study also observed that older age and hypertension were associated with elevated Aix.

(Poly)phenols (PP) are known to have antioxidant effects, and therefore may be beneficial against ROS.

PP are secondary plant metabolites containing one or more phenolic rings in their structure, and with a mass ranging from 300 to 3000 Da, and even up to 20 000 Da for large compounds. More than 8000 different types of PP have been identified in plants.

They are very abundant in fruits and vegetables, and plant foods and beverages such as coffee, tea, nuts, olive oil, soy products or cocoa. In recent years, these compounds have attracted a lot of attention and have been widely investigated due to their potential health benefits. Epidemiological and clinical studies have shown that PP have the potential to modulate physiopathological conditions and thus reduce the risk of chronic diseases such as CVD and dementia.

PP are one of the most abundant and major groups of phytochemicals, which also includes terpenoids-such as carotenoids-, alkaloids and sulphur compounds. They are synthesized by plants to protect them against UV-mediated oxidative stress and strengthen the cell wall, to repel herbivores and infections, and to attract pollinators.

PP are naturally found on their glycoside form and less frequently as an aglycone or genin (i.e., without the glycosyl moiety). They are classified into different groups based on the number of phenyl rings and their structure. Thus, PP can be divided in two major groups: flavonoid and non-flavonoids.

Flavonoids are chemically related to a 15-carbon skeleton structure, which consists of two phenyl rings, A and B, and one heterocyclic ring containing oxygen named C. This structure is also known as “C6-C3-C6”. Usually, the B ring is bound to position no 2 of the C ring, but it can also be found in position no 3, as is the case of isoflavones. Flavonoids are divided in 7 sub-classes: flavones, flavanones, anthocyanins, flavonols, flavan-3-ols, isoflavones, and dihydrochalcones.

The non-flavonoids family include stilbenes, lignans, phenolic acids, and other PP, such as tyrosols, pyrogallols, or hydroxycoumarins (Manach et al. 2004). Phenolic acids are divided five subgroups, i.e. hydroxyphenylpropanoic-, hydroxyphenylacetic-, into hydroxyphenylpentanoic-, hydroxycinnamic- and hydroxybenzoic-acids (Phenol Explorer).

Most PP are orally ingested; therefore, the internal digestion of any PP will have a direct effect on the activity of the ingested PP.

Often considered as a “second brain”, the microbiome is a large ecosystem composed of trillions of bacteria which interacts with the whole organism during all the life of an individual.

The gut microbiota exerts an important role in the bioavailability of PP as the presence of high molecular weight compounds associated with the relatively low absorption of phenolic compounds in general favour the interaction of the latter with colonic bacteria.

When reaching the large intestine, a bidirectional relationship will develop between PP and microbiota. Indeed, while PP can modulate the composition and diversity of the microbiome, intestinal bacteria catabolize PP to produce smaller compounds that are usually more active and which present a better absorption than the original metabolite.

Therefore, the microbiome can play an important role in maintaining physiological functions of the body. Dysbiosis of the microbiome can lead to various disorders. Microbe-based therapies can be used for maintenance of gut health and treatment of microbiome-related disorders. For example, it was reported in Jing Li et al that gut microbiota dysbiosis contributes to the development of hypertension (Li, J., Zhao, et al. 2017. ‘Gut microbiota dysbiosis contributes to the development of hypertension’, Microbiome, 5(1), pp. 1-19.)

Aronia melanocarpa (Aronia) is a berry belonging to the Rosaceae family. Also known as “black chokeberry” due to its astringent taste, the berry is originally native from Northern America. However, Aronia is nowadays found and cultivated in Central and Eastern Europe (USDA), and, since its introduction, various cultivars have been created, such as Nero, Viking or Aron which present bigger berries and a better resistance compared to the original cultivar. Aronia berries are a rich source of PP and are considered one of the highest sources of PP among berries in general. Main PP found are in the berry are represented by procyanidins, anthocyanins, and phenolic acids.

The listing or discussion of an apparently prior-published document in this specification should not necessarily be taken as an acknowledgement that the document is part of the state of the art or is common general knowledge.

### DISCLOSURE OF THE INVENTION

It has been surprisingly and unexpectedly found by the present inventers that Aronia berry extracts can treat and/or prevent cardiovascular disease (CVD) and/or gut dysbiosis.

Therefore, the present invention provides a composition for use in treating and/or preventing cardiovascular disease, wherein the composition comprises an extract obtained or obtainable from Aronia melanocarpa.

The present invention also provides a composition for use in treating and/or preventing gut dysbiosis, wherein the composition comprises an extract obtained or obtainable from Aronia melanocarpa.

The present invention also provides a method for treating and/or preventing cardiovascular disease, wherein the method comprises the administration of a composition comprising an extract obtained or obtainable from Aronia melanocarpa.

The present invention also provides a method for treating and/or preventing gut dysbiosis, wherein the method comprises the administration of a composition comprising an extract obtained or obtainable from Aronia melanocarpa.

The invention may also provide the use of a composition comprising an extract obtained or obtainable from Aronia melanocarpa in the manufacture of a medicament for the treatment and/or prevention of cardiovascular disease and/or gut dysbiosis.

As used herein, treating cardiovascular disease may include at least one of:
(a) Treating and/or preventing hypertension; and/or
(b) Treating or preventing pre-hypertension, and/or
(b) Reducing blood pressure; and/or
(c) Reducing arterial stiffness.

For example, the present invention may provide a composition for use in treating and/or preventing hypertension, reducing blood pressure and/or reducing arterial stiffness, wherein the composition comprises an extract obtained or obtainable from Aronia melanocarpa. The invention may also provide a method for treating and/or preventing hypertension, reducing blood pressure and/or reducing arterial stiffness, wherein the method comprises the administration of a composition comprising an extract obtained or obtainable from Aronia melanocarpa to a subject in need thereof.

The invention may provide the use of a composition comprising an extract obtained or obtainable from Aronia melanocarpa in the manufacture of a medicament for the treatment and/or prevention of hypertension, for the treatment and/or prevention of pre-hypertension, reducing blood pressure and/or reducing arterial stiffness.

Arterial stiffness has been shown to be associated with an increased risk of cardiovascular disease events and cardiovascular mortality. For example, a 10% increase in Aix was shown to lead to an increase of 48% in the risk of cardiovascular mortality (London et al 2001 J Spinal Cord Medicine 32:72-78. Accordingly, it will be clear that a reduction in arterial stiffness is taken to indicate treatment or prevention of CVD or CVD events.

As used herein, treating gut dysbiosis may include at least one of:
(a) Increasing microbiome diversity as measured by faecal microbiome gene count;
(b) Increasing levels of beneficial bacterial, including but not limited to one or more of Faecalibacterium prausnitzii 2, Lawsonibacter asaccharolyticus, Intestinimonas butyriciproducens, Faecalibacterium, Roseburia intestinalis, and/or Eggerthella lenta and/or
c) decreasing levels of pathogenic microorganisms or microorganisms identified as not beneficial or less beneficial for one or more conditions

For example, the present invention may provide a composition for use in increasing microbiome diversity as measured by faecal microbiome gene count and/or increasing levels of beneficial bacterial, including but not limited to one or more of Clostridiales bacterium, Oscillibacter sp. Firmicutes bacterium CAG 103, Lawsonibacter asaccharolyticus, Oscillospirales 5, Clostridium sp., Butyricimonas faecihominis, Turicibacter sanguinis, Bacteroides dorei, Oscillospiraceae, Bacteroides xylanisolvens, Ruminococcus sp./Blautia sp., Dialister invisus, Flavonifractor sp., Clostridium sp., Faecalibacterium prausnitzii 2, Christensenellales, Blautia A, Intestinimonas butyriciproducens, Faecalibacterium, Roseburia intestinalis and/or Eggerthella lenta.

In a preferred embodiment, the bacteria increased include one or more of Faecalibacterium prausnitzii 2, Lawsonibacter asaccharolyticus, Intestinimonas butyriciproducens, Faecalibacterium, Roseburia intestinalis and/or Eggerthella lenta.

In a preferred embodiment, the increasing levels of beneficial bacterial include one or more of Faecalibacterium prausnitzii 2, Lawsonibacter asaccharolyticus and/or Intestinimonas butyriciproducens.

The present invention may also provide a method for increasing microbiome diversity as measured by faecal microbiome gene count and/or increasing levels of beneficial bacterial, including but not limited to one or more of Faecalibacterium prausnitzii 2, Lawsonibacter asaccharolyticus Intestinimonas butyriciproducens Faecalibacterium, Roseburia intestinalis and/or Eggerthella lenta, wherein the method comprises the administration of a composition comprising an extract obtained or obtainable from Aronia melanocarpa to a subject in need thereof. In a preferred embodiment, the increasing levels of beneficial bacterial include one or more of Faecalibacterium prausnitzii 2, Lawsonibacter asaccharolyticus and/or Intestinimonas butyriciproducens

In some embodiments the faecal microbiome diversity is increased by at least 2% such as at least 3%, 4%, 5%, 10%, 15%, 20%, 25%, 30%, 35%, 40%, 45% or at least 50% following treatment with a composition of the invention relative to the faecal microbiome diversity of the subject prior to administration of the composition.

Such compositions for use or methods are hereinafter referred to as the “compositions for use of the invention” or “methods of the invention”.

Hypertension, also known as high or raised blood pressure, is a condition in which the blood vessels have persistently raised pressure. Blood is carried from the heart to all parts of the body in the vessels. Each time the heart beats, it pumps blood into the vessels. Blood pressure is created by the force of blood pushing against the walls of blood vessels (arteries) as it is pumped by the heart. The higher the pressure, the harder the heart has to pump.

Typically, hypertension is defined as having a systolic blood pressure of 130 or more, or 140 or more and/or a diastolic blood pressure of 80 or more, or 90 or more. Pre-hypertension is defined as having a systolic blood pressure between 120 and 130 or between 120 and 140 and/or a diastolic blood pressure between 80 and 90.

In certain embodiments, treating high blood pressure is defined as reducing at least 2 points of mm Mercure, at least 3, 4, 5, 6, 7, 8, 9, 10, 11 point of mm in respect to the of the pre-treatment value after a certain treatment period. In certain embodiments, treating pre-hypertension pressure is defined as reducing at least 2 points of mm Mercure, at least 3, 4, 5, 6, 7, 8, 9, 10, 11 points of mm in respect to the of the pre-treatment value after a certain treatment period.

Arterial stiffness is typically measured using Pulse wave velocity (PWV) and/or Augmentation index (Aix).

Therefore, in the present invention, reducing arterial stiffness may be measured by determining the PWV and/or AIx. A reduction in PWV and/or AIx indicates a reduction in arterial stiffness.

In some embodiments of the invention, treatment with a composition of the invention results in a reduction in arterial stiffness of at 2% or greater, for example at least 3%, at least 4%, at least 5%, at least 10%, 15%, 20%, 25%, 30% or more relative to the arterial stiffness of the subject prior to treatment with the composition. In some embodiments the decrease in arterial stiffness is 2% or greater, for example at least 3%, at least 4%, at least 5%, or at least 10%, 15%, 20%, 25%, 30% is relative to the arterial stiffness of a control group of subjects. In some embodiments the control group is represented by a population of individuals known to have high arterial stiffness, for example a population of individuals that have cardiovascular disease. The skilled person is able to select an appropriate composition of control subjects.

Microbiome diversity is dependent on the number of genetically different bacterial species present in the relevant biome. In the present invention, the biome is the gut microbiome, for example the gut microbiome as detectable in the faeces. Therefore, increasing microbiome diversity as used in the present invention is intended to mean increasing the number of genetically different bacterial species in the gut as measured by faecal microbiome gene count. For example, in some embodiments increasing microbial diversity in the gut includes increasing the number of genetically different bacterial species in the gut by at least 1%, or at least 2%, 3%, 4%, 5%, 10%, 15%, 20%, 25%, 30%, 35% or by at least 40% relative to the microbial diversity in the gut of the individual prior to treatment with the composition of the invention.

In some embodiments, the faecal microbial diversity of a subject is increased by at least 1%, or at least 2%, 3%, 4%, 5%, 10%, 15%, 20%, 25%, 30%, 35% or by at least 40% relative to the microbial diversity present in a population of samples obtained from subjects known to have a low microbial diversity.

In some embodiments increasing microbial diversity in the gut includes increasing the gene count in a faecal sample of the subject by at least 1%, or at least 2%, 3%, 4%, 5%, 10%, 15%, 20%, 25%, 30%, 35% or by at least 40% relative to the gene count in a faecal sample taken from the individual prior to treatment with the composition of the invention.

In some embodiments increasing microbial diversity in the gut is achieved when the microbial diversity in a faecal sample obtained from the subject is the same as, or substantially similar to (i.e. within a statistically appropriate range or), the mean microbial diversity of a healthy population of subjects.

In some embodiments increasing microbial diversity in the gut includes increasing the gene count in a faecal sample of the subject by at least 1%, or at least 2%, 3%, 4%, 5%, 10%, 15%, 20%, 25%, 30%, 35% or by at least 40% relative to the gene count in a population of faecal samples taken from a population of subjects known to have a low microbial diversity.

In some embodiments increasing microbial diversity in the gut is achieved when the gene count in a faecal sample obtained from the subject is the same as, or substantially similar to (i.e. within a statistically appropriate range or), the mean gene count of a healthy population of subjects.

In some embodiments the mean gene count of a healthy population of subjects is about 370.000 or at least 370.000, about 380.000 or at least 380000, 400,000 or at least 400,000, for example at least 450,000, 500,000, 550,000 or at least 600,000.

The type of bacteria present in the gut microbiome also plays an important part in the health of the gut. In the present invention, it has been found that increasing levels of beneficial bacterial, including but not limited to one or more of Faecalibacterium prausnitzii 2, Lawsonibacter asaccharolyticus and Intestinimonas butyriciproducens Faecalibacterium, Roseburia intestinalis and/or Eggerthella lenta improves gut heath and dysbiosis.

Without being bound to any theory the increase in gene count and the increase in certain beneficial bacteria may positively influence the conditions of the gut and can be used as biomarkers of certain gut conditions, which may predict certain diseases. For example, Faecalibacterium prausnitzii has been found to be a good biomarker for discriminating Crohn's disease (CD) and colorectal cancer (CRC) from healthy subjects (Lopez-Siles et al, The ISME Journal, (2017), 11, 841-852).

The present inventors have surprisingly and unexpected found that increasing levels of Faecalibacterium prausnitzii 8, Acutalibacteraceae 3, Firmicutes bacterium CAG 103 and/or Bifidobacterium adolescentis can be used to predict those pre-hypertensive subjects that may response to treatment with a composition of the invention as discussed previously.

It has also been surprisingly found by the present inventors that treating cardiovascular disease and/or gut dysbiosis with an extract obtained or obtainable from Aronia melanocarpa as defined above, is more beneficial in subpopulations of mammals (i.e. humans).

Populations of subjects that were found to be most likely to respond to treatment with the composition of the invention were those which had a low faecal microbiome gene count and relatively high abundances of Faecalibacterium prausnitzii 8, Acutalibacteraceae 3, Firmicutes bacterium CAG 103 and/or Bifidobacterium adolescentis.

Accordingly, in one embodiment the invention provides:
a method for determining whether a subject is likely to respond to treatment with a composition that comprises an extract obtained or obtainable from Aronia melanocarpa, wherein the method comprises:
   a) determining the faecal microbiome gene count in a sample of faeces obtained from the subject; and/or
   b) determining the abundance of any 1, 2, 3 or all of:
      i) Faecalibacterium prausnitzii 8 in a sample of faeces obtained from the subject;
      ii) Acutalibacteraceae 3 in a sample of faeces obtained from the subject;
      iii) Firmicutes bacterium CAG 103 in a sample of faeces obtained from the subject; and/or
      iv) Bifidobacterium adolescentis in a sample of faeces obtained from the subject.

It will be clear that in some instances it is necessary to compare the gene count and/or abundance of the particular microbes to a control sample or samples, or to standardized value, so that the skilled person can determine whether a particular subject is likely to be a responder to treatment, or not.

Accordingly in some embodiments the method for determining whether a subject is likely to respond to treatment further comprises
a) comparing the faecal microbiome gene count in the sample of faeces obtained from the subject with the faecal microbiome gene count in at least a first control sample or population of control samples; and/or
b) comparing the abundance of:
   i) Faecalibacterium prausnitzii 8 in a sample of faeces obtained from the subject with the abundance of Faecalibacterium prausnitzii 8 in at least a first control sample or population of control samples;
   ii) Acutalibacteraceae 3 in a sample of faeces obtained from the subject with the abundance of Acutalibacteraceae 3 in at least a first control sample or population of control samples;
   iii) Firmicutes bacterium CAG 103 in a sample of faeces obtained from the subject with the abundance of Firmicutes bacterium CAG 103 in at least a first control sample or population of control samples; and/or
   iv) Bifidobacterium adolescentis in a sample of faeces obtained from the subject with the abundance of Bifidobacterium adolescentis in at least a first control sample or population of control samples.

The skilled person is well placed to choose appropriate control samples, and the skilled person will be well versed in positive and negative control samples. The discussion below relating to control samples applies to other embodiments disclosed herein, for example in the context of arterial stiffness.

In some embodiments the control sample is a single control sample, for example taken from a single individual. In other preferred examples the control sample is actually taken from a population of control samples. The skilled person will appreciate that the statistical power of a method increases with the number of independent samples involved. Accordingly, the skilled person is able to select an appropriate number of control samples to use. For example, the population of control samples may comprise at least 5, 10, 20, 30, 40, 50, 60, 70, 80, 90, 100, 150, 200, 250, 300, 500 or at least 1000 different control samples.

The skilled person will also be aware of appropriate statistical methods that can be employed on a population of control samples to obtain a single value, for example a mean value, that can be directly compared to a test value. Accordingly, in some embodiments the control sample or samples is actual a single mean value of the relevant parameter (e.g. gene count) obtained from a population of samples. The skilled person is then able to compare the test value (e.g. test gene count) to the mean gene count from the relevant control population, and so is able to determine whether the test sample (e.g. gene count) is within a statistically significant range of the control value, or is outside this range.

The skilled person will appreciate that is necessary to compare a test value or sample to at least a positive control value or samples, or at least a negative control value or samples, or preferably to both a positive and negative control value or samples.

The inventors have surprisingly found that subjects with a low gene count, and/or higher relative abundance of particular microbial species are more likely to respond to treatment with the composition of the present invention. Accordingly in this scenario a positive sample or population of samples would be a sample or set of samples taken from subjects that are known to respond to treatment with the composition of the invention. It is important that the samples from the positive control population that are to be used in generating a positive control sample set, or mean value for instance, are those samples that were taken from the subjects prior to administration of the composition.

Similarly, for the negative control samples, a negative sample or population of samples would be a sample or set of samples taken from subjects that are known to not respond to treatment with the composition of the invention. As above, it is important that the samples from the negative control population that are to be used in generating a negative control sample set, or mean value for instance, are those samples that were taken from the subjects prior to administration of the composition.

Accordingly, in one embodiment the at least a first control sample or population of control samples is a negative control sample or population or control samples or is for example statistically relevant value obtained from the population of control samples, for example a mean value.

In some embodiments the negative control sample or population of control samples are faecal samples taken from one or more than one negative control subjects that have been determined to not respond to treatment with a composition that comprises an extract obtained or obtainable from Aronia melanocarpa, wherein the samples were taken from the one or more than one control subjects prior to treatment with a composition that comprises an extract obtained or obtainable from Aronia melanocarpa.

In some embodiments the at least a first control sample or population of control samples is a positive control sample or population or control samples, or is for example statistically relevant value obtained from the population of control samples, for example a mean value.

In some embodiments the positive control sample or population of control samples are faecal samples taken from one or more than one positive control subjects that have been shown to respond to treatment with a composition that comprises an extract obtained or obtainable from Aronia melanocarpa, wherein the samples were taken from the one or more than one control subjects prior to treatment with a composition that comprises an extract obtained or obtainable from Aronia melanocarpa,

In some embodiments the faecal microbiome gene count of the sample obtained from the subject is compared to:
a) the faecal microbiome gene count in a negative control sample or population or control samples, or statistically relevant value obtained from the population of control samples, for example a mean value, for example wherein the negative control sample or population of control samples are faecal samples taken from one or more than one negative control subjects that have been shown to not respond to treatment with a composition that comprises an extract obtained or obtainable from Aronia melanocarpa, wherein the samples were taken from the one or more than one negative control subjects prior to treatment with a composition that comprises an extract obtained or obtainable from Aronia melanocarpa; and/or
b) the faecal microbiome gene count in a positive control sample or population or control samples or statistically relevant value obtained from the population of control samples, for example a mean value, for example wherein the positive control sample or population of control samples are faecal samples taken from one or more than one positive control subjects that have been shown to respond to treatment with a composition that comprises an extract obtained or obtainable from Aronia melanocarpa, wherein the samples were taken from the one or more than one positive control subjects prior to treatment with a composition that comprises an extract obtained or obtainable from Aronia melanocarpa.

In some embodiments:
a) where the faecal microbiome gene count of the sample obtained from the subject is substantially similar to or higher than the faecal microbiome gene count of the negative control sample or samples or statistically relevant value obtained from the population of control samples, for example a mean value, the subject is considered unlikely to respond to treatment with a composition that comprises an extract obtained or obtainable from Aronia melanocarpa,
for example wherein where the faecal microbiome gene count of the sample obtained from the subject is at least 5% greater, such as at least 10% greater, 15%, 20%, 25%, 30%, 35%, 40%, 50%, 60%, 70%, 80%, 90%, at least 100% greater, such as at least 200% greater than the faecal microbiome gene count of the negative control sample or samples, the subject is considered to be unlikely to respond to treatment with a composition that comprises an extract obtained or obtainable from Aronia melanocarpa;
   and/or
b) where the faecal microbiome gene count in the test sample is substantially similar to or lower than the faecal microbiome gene count of the positive control sample or samples or statistically relevant value obtained from the population of control samples, for example a mean value, the subject is considered likely to respond to treatment with a composition that comprises an extract obtained or obtainable from Aronia melanocarpa,
for example wherein where the faecal microbiome gene count of the sample obtained from the subject is at least is at least 5% lower, such as at least 10% lower, 15%, 20%, 25%, 30%, 35%, 40%, 50%, 60%, 70%, 80%, 90%, at least 100% lower, such as at least 200% lower greater than the faecal microbiome gene count of the positive control sample or samples, the subject is considered to be likely to respond to treatment with a composition that comprises an extract obtained or obtainable from Aronia melanocarpa.

The skilled person is able to determine whether a particular test value is significantly different to the positive or negative control samples and is able to determine whether a particular test sample should be classified as coming from a responder or non-responder.

In some embodiments:
a) the abundance of:
   i) Faecalibacterium prausnitzii 8 in a sample of faeces obtained from the subject is compared to the abundance of Faecalibacterium prausnitzii 8 in at least a first control sample or population of control samples or statistically relevant value obtained from the population of control samples, for example a mean value;
   ii) Acutalibacteraceae 3 in a sample of faeces obtained from the subject is compared to the abundance of Acutalibacteraceae 3 in at least a first control sample or population of control sample or statistically relevant value obtained from the population of control samples, for example a mean value;
   iii) Firmicutes bacterium CAG 103 in a sample of faeces obtained from the subject is compared to the abundance of Firmicutes bacterium CAG 103 in at least a first control sample or population of control samples or statistically relevant value obtained from the population of control samples, for example a mean value; and/or
   iv) Bifidobacterium adolescentis in a sample of faeces obtained from the subject is compared to the abundance of Bifidobacterium adolescentis in at least a first control sample or population of control samples or statistically relevant value obtained from the population of control samples, for example a mean value;
   and wherein the control sample or population of control samples are a negative control sample or population or control samples or statistically relevant value obtained from the population of control samples, for example a mean value
   wherein the negative control sample or population of control samples are faecal samples taken from one or more than one negative control subjects that have been shown to not respond to treatment with a composition that comprises an extract obtained or obtainable from Aronia melanocarpa, wherein the samples were taken from the one or more than one positive control subjects prior to treatment with a composition that comprises an extract obtained or obtainable from Aronia melanocarpa; and/or
b) the abundance of:
   i) Faecalibacterium prausnitzii 8 in a sample of faeces obtained from the subject is compared to the abundance of Faecalibacterium prausnitzii 8 in at least a first control sample or population of control samples or statistically relevant value obtained from the population of control samples, for example a mean value;
   ii) Acutalibacteraceae 3 in a sample of faeces obtained from the subject is compared to the abundance of Acutalibacteraceae 3 in at least a first control sample or population of control samples or statistically relevant value obtained from the population of control samples, for example a mean value;
   iii) Firmicutes bacterium CAG 103 in a sample of faeces obtained from the subject is compared to the abundance of Firmicutes bacterium CAG 103 in at least a first control sample or population of control samples or statistically relevant value obtained from the population of control samples, for example a mean value; and/or
   iv) Bifidobacterium adolescentis in a sample of faeces obtained from the subject is compared to the abundance of Bifidobacterium adolescentis in at least a first control sample or population of control samples or statistically relevant value obtained from the population of control samples, for example a mean value;
   and wherein the control sample or population of control samples are a positive control sample or population or control samples,
   wherein the positive control sample or population of control samples are faecal samples taken from one or more than one positive control subjects that have been shown to respond to treatment with a composition that comprises an extract obtained or obtainable from Aronia melanocarpa, wherein the samples were taken from the one or more than one positive control subjects prior to treatment with a composition that comprises an extract obtained or obtainable from Aronia melanocarpa,

In some embodiments:
a) where the abundance of Faecalibacterium prausnitzii 8 in a sample of faeces obtained from the subject is substantially similar to or higher than the abundance of Faecalibacterium prausnitzii 8 in a positive control sample or samples or statistically relevant value obtained from the population of control samples, for example a mean value, the subject is considered likely to respond to treatment with a composition that comprises an extract obtained or obtainable from Aronia melanocarpa, optionally wherein where the abundance of Faecalibacterium prausnitzii 8 in the sample obtained from the subject is at least 5% greater, such as at least 10% greater, 15%, 20%, 25%, 30%, 35%, 40%, 50%, 60%, 70%, 80%, 90%, at least 100% greater, such as at least 200% greater than the abundance of Faecalibacterium prausnitzii 8 in a positive control sample or samples or statistically relevant value obtained from the population of control samples, for example a mean value, the subject is considered likely to respond to treatment with a composition that comprises an extract obtained or obtainable from Aronia melanocarpa;
b) where the abundance of Acutalibacteraceae 3 in a sample of faeces obtained from the subject is substantially similar to or higher than the abundance of Acutalibacteraceae 3 in a positive control sample or samples or statistically relevant value obtained from the population of control samples, for example a mean value, the subject is considered likely to respond to treatment with a composition that comprises an extract obtained or obtainable from Aronia melanocarpa, optionally wherein where the abundance of Acutalibacteraceae 3 in the sample obtained from the subject is at least at least 5% greater, such as at least 10% greater, 15%, 20%, 25%, 30%, 35%, 40%, 50%, 60%, 70%, 80%, 90%, at least 100% greater, such as at least 200% greater than the abundance of Acutalibacteraceae 3 in a positive control sample or samples or statistically relevant value obtained from the population of control samples, for example a mean value, the subject is considered likely to respond to treatment with a composition that comprises an extract obtained or obtainable from Aronia melanocarpa;
c) where the abundance of Firmicutes bacterium CAG 103 in a sample of faeces obtained from the subject is substantially similar to or higher than the abundance of Firmicutes bacterium CAG 103 in a positive control sample or samples or statistically relevant value obtained from the population of control samples, for example a mean value, the subject is considered likely to respond to treatment with a composition that comprises an extract obtained or obtainable from Aronia melanocarpa, optionally wherein where the abundance of Firmicutes bacterium CAG 103 in the sample obtained from the subject is at least 5% greater, such as at least 10% greater, 15%, 20%, 25%, 30%, 35%, 40%, 50%, 60%, 70%, 80%, 90%, at least 100% greater, such as at least 200% greater than the abundance of Firmicutes bacterium CAG 103 in a positive control sample or samples or statistically relevant value obtained from the population of control samples, for example a mean value, the subject is considered likely to respond to treatment with a composition that comprises an extract obtained or obtainable from Aronia melanocarpa;
   and/or
d) where the abundance of Bifidobacterium adolescentis in a sample of faeces obtained from the subject is substantially similar to or higher than the abundance of Bifidobacterium adolescentis in a positive control sample or samples or statistically relevant value obtained from the population of control samples, for example a mean value, the subject is considered likely to respond to treatment with a composition that comprises an extract obtained or obtainable from Aronia melanocarpa, optionally wherein where the abundance of Bifidobacterium adolescentis in the sample obtained from the subject is at least 5% greater, such as at least 10% greater, 15%, 20%, 25%, 30%, 35%, 40%, 50%, 60%, 70%, 80%, 90%, at least 100% greater, such as at least 200% greater than the abundance of Bifidobacterium adolescentis in a positive control sample or samples or statistically relevant value obtained from the population of control samples, for example a mean value, the subject is considered likely to respond to treatment with a composition that comprises an extract obtained or obtainable from Aronia melanocarpa;
e) where the abundance of Faecalibacterium prausnitzii 8 in a sample of faeces obtained from the subject is substantially similar to or lower than the abundance of Faecalibacterium prausnitzii 8 in a negative control sample or samples or statistically relevant value obtained from the population of control samples, for example a mean value, the subject is considered unlikely to respond to treatment with a composition that comprises an extract obtained or obtainable from Aronia melanocarpa, optionally wherein where the abundance of Faecalibacterium prausnitzii 8 in the sample obtained from the subject is at least 5% lower, such as at least 10% greater, 15%, 20%, 25%, 30%, 35%, 40%, 50%, 60%, 70%, 80%, 90%, at least 100% lower, such as at least 200% lower than the abundance of Faecalibacterium prausnitzii 8 in a negative control sample or samples or statistically relevant value obtained from the population of control samples, for example a mean value, the subject is considered unlikely to respond to treatment with a composition that comprises an extract obtained or obtainable from Aronia melanocarpa;
f) where the abundance of Acutalibacteraceae 3 in a sample of faeces obtained from the subject is substantially similar to or lower than the abundance of Acutalibacteraceae 3 in a negative control sample or samples or statistically relevant value obtained from the population of control samples, for example a mean value, the subject is considered unlikely to respond to treatment with a composition that comprises an extract obtained or obtainable from Aronia melanocarpa, optionally wherein where the abundance of Acutalibacteraceae 3 in the sample obtained from the subject is at least 5% lower, such as at least 10% greater, 15%, 20%, 25%, 30%, 35%, 40%, 50%, 60%, 70%, 80%, 90%, at least 100% lower, such as at least 200% lower than the abundance of Acutalibacteraceae 3 in a negative control sample or samples or statistically relevant value obtained from the population of control samples, for example a mean value, the subject is considered unlikely to respond to treatment with a composition that comprises an extract obtained or obtainable from Aronia melanocarpa;
g) where the abundance of Firmicutes bacterium CAG 103 in a sample of faeces obtained from the subject is substantially similar to or lower than the abundance of Firmicutes bacterium CAG 103 in a negative control sample or samples or statistically relevant value obtained from the population of control samples, for example a mean value, the subject is considered unlikely to respond to treatment with a composition that comprises an extract obtained or obtainable from Aronia melanocarpa, optionally wherein where the abundance of Firmicutes bacterium CAG 103 in the sample obtained from the subject is at least 5% lower, such as at least 10% greater, 15%, 20%, 25%, 30%, 35%, 40%, 50%, 60%, 70%, 80%, 90%, at least 100% lower, such as at least 200% lower than the abundance of Firmicutes bacterium CAG 103 in a negative control sample or samples or statistically relevant value obtained from the population of control samples, for example a mean value, the subject is considered unlikely to respond to treatment with a composition that comprises an extract obtained or obtainable from Aronia melanocarpa;
   and/or
h) where the abundance of Bifidobacterium adolescentis in a sample of faeces obtained from the subject is substantially similar to or lower than the abundance of Bifidobacterium adolescentis in in a negative control sample or samples or statistically relevant value obtained from the population of control samples, for example a mean value, the subject is considered unlikely to respond to treatment with a composition that comprises an extract obtained or obtainable from Aronia melanocarpa, optionally wherein where the abundance of Bifidobacterium adolescentis in the sample obtained from the subject is at least 5% lower, such as at least 10% greater, 15%, 20%, 25%, 30%, 35%, 40%, 50%, 60%, 70%, 80%, 90%, at least 100% lower, such as at least 200% lower than the abundance of Bifidobacterium adolescentis in a negative control sample or samples or statistically relevant value obtained from the population of control samples, for example a mean value, the subject is considered unlikely to respond to treatment with a composition that comprises an extract obtained or obtainable from Aronia melanocarpa.

In some preferred embodiments, the abundance of each of the above microbes is determined. Accordingly in some embodiments:
i) the abundance of Faecalibacterium prausnitzii 8 in a sample of faeces obtained from the subject is compared to the abundance of Faecalibacterium prausnitzii 8 in at least a first control sample or population of control samples or statistically relevant value obtained from the population of control samples, for example a mean value;
ii) the abundance of Acutalibacteraceae 3 in a sample of faeces obtained from the subject is compared to the abundance of Acutalibacteraceae 3 in at least a first control sample or population of control samples or statistically relevant value obtained from the population of control samples, for example a mean value;
iii) the abundance of Firmicutes bacterium CAG 103 in a sample of faeces obtained from the subject is compared to the abundance of Firmicutes bacterium CAG 103 in at least a first control sample or population of control samples or statistically relevant value obtained from the population of control samples, for example a mean value; and
iv) the abundance of Bifidobacterium adolescentis in a sample of faeces obtained from the subject is compared to the abundance of Bifidobacterium adolescentis in at least a first control sample or population of control samples or statistically relevant value obtained from the population of control samples, for example a mean value.

The abundance of Bifidobacterium adolescentis is considered to be particularly useful indicating whether a subject will respond to the treatment or not. Accordingly, in one embodiment the method comprises determining the abundance of Bifidobacterium adolescentis in the faecal sample obtained from the subject and comparing that to the abundance of Bifidobacterium adolescentis in:
one or more negative control samples or statistically relevant value obtained from the population of control samples, for example a mean value; and/or
one or more positive control samples or statistically relevant value obtained from the population of control samples, for example a mean value.

In some embodiments, the method for determining whether a subject is likely to respond to treatment with the composition of the invention simply involves determining the faecal microbiome gene count in the sample of faeces obtained from the subject. In some embodiments where the faecal microbiome gene count is less than 400,000, optionally less than 375,000, optionally less than 350,000, 300,000, 275,000, 250,000, 225,000, 200,000, 175,000, or less than 150,000, then the subject is considered likely to respond to treatment with the composition of the invention.

As described elsewhere herein, one method for determining the microbiome gene count is by shotgun sequencing. Accordingly, in some embodiments of any aspect or embodiment of the invention the faecal microbiome gene count in a sample of faeces is determined by shotgun sequencing.

Likewise, in some embodiments of any aspect or embodiment of the invention, determining the abundance of:
i) Faecalibacterium prausnitzii 8 in a sample of faeces obtained from the subject;
ii) Acutalibacteraceae 3 in a sample of faeces obtained from the subject;
iii) Firmicutes bacterium CAG 103 in a sample of faeces obtained from the subject; and/or
iv) Bifidobacterium adolescentis in a sample of faeces obtained from the subject is performed using shotgun sequencing.

In some embodiments, by respond to treatment with a composition of the invention, is intended to mean respond by showing a reduction in blood pressure. Accordingly in one embodiment a response to treatment is indicated by a reduction in blood pressure.

For example, the invention provides a method for determining whether a composition that comprises an extract obtained or obtainable from Aronia melanocarpa is likely to reduce the blood pressure of a particular subject, wherein the method comprises the method for determining whether a subject is likely to respond to treatment with the composition of the invention as described herein.

In some embodiments of the method for determining whether a composition that comprises an extract obtained or obtainable from Aronia melanocarpa is likely to reduce the blood pressure of a particular subject and/or the method for determining whether a subject is likely to respond to treatment with the composition, the subject is prehypertensive, for example the subject has:
a systolic blood pressure of at least 120 mmHg, optionally at least 130 or at least 140 mmHg; and/or a systolic blood pressure of between 120 mmHg and 130 mmHg, or between 120 mmHg and 140 mmHg, and/or
a diastolic blood pressure of at least 80 mmHg, optionally at least 90, mmHg; and/or a diastolic blood pressure of between 80 mmHg and 90 mmHg.

The invention also therefore provides a composition for use in treating and/or preventing cardiovascular disease and/or gut dysbiosis in a subject, wherein the composition comprises an extract obtained or obtainable from Aronia melanocarpa and wherein the subject has been determined to be likely to respond to treatment with the composition according to any the methods of the invention.

The invention also provides a method for treating and/or preventing cardiovascular disease and/or gut dysbiosis in a subject, wherein the method comprises the administration of a composition comprising an extract obtained or obtainable from Aronia melanocarpa to a subject in need thereof, and wherein the subject has been determined to be likely to respond to treatment with the composition according to any the methods of the invention.

The invention also therefore provides a composition for use in treating hypertension, prehypertension and/or reducing high blood pressure in a subject, wherein the composition comprises an extract obtained or obtainable from Aronia melanocarpa and wherein the subject has been determined to be likely to respond to treatment with the composition according to any the methods of the invention.

The invention also provides a method for treating hypertension, prehypertension and/or reducing high blood pressure in a subject, wherein the method comprises the administration of a composition comprising an extract obtained or obtainable from Aronia melanocarpa to a subject in need thereof, and wherein the subject has been determined to be likely to respond to treatment with the composition according to any the methods of the invention.

The present invention may also provide a composition for use in treating gut dysbiosis in a mammal (i.e. human) having a faecal microbiome gene count of less than 400,000, optionally less than 375,000, optionally less than 350,000, 300,000, 275,000, 250,000, 225,000, 200,000, 175,000, or less than 150,000, wherein the composition comprises an extract obtained or obtainable from Aronia melanocarpa.

The present invention may also provide a method for treating gut dysbiosis in a mammal (i.e. human) having a faecal microbiome gene count of less than 400,000, optionally less than 375,000, optionally less than 350,000, 300,000, 275,000, 250,000, 225,000, 200,000, 175,000, or less than 150,000, wherein the method comprises the administration of a composition comprising an extract obtained or obtainable from Aronia melanocarpa.

The present invention may also provide the use of a composition comprising an extract obtained or obtainable from Aronia melanocarpa in a method of manufacture of a medicament for use in treating gut dysbiosis in a mammal (i.e. human) having a faecal microbiome gene count similar to or lower than the faecal microbiome gene count of the positive control sample or samples or statistically relevant value obtained from the population of control samples, for example a mean value, for example wherein the faecal microbiome gene count of the sample obtained from the subject is at least is at least 5% lower, such as at least 10% lower, 15%, 20%, 25%, 30%, 35%, 40%, 50%, 60%, 70%, 80%, 90%, at least 100% lower, such as at least 200% lower greater than the faecal microbiome gene count of the positive control sample or samples,

The present invention may also provide the use of a composition comprising an extract obtained or obtainable from Aronia melanocarpa in a method of manufacture of a medicament for use in treating gut dysbiosis in a mammal (i.e. human) having a faecal microbiome gene count of less than 400,000, optionally less than 375,000, optionally less.

The inventors of the present invention have surprisingly found that the supplementation of Aronia melanocarpa results in an increase of the microbiome diversity in the subjects and more specifically the bacteria shown in FIG. 9.

The present invention may also provide a composition for use in increasing microbiome diversity as measured by faecal microbiome gene count and/or increasing levels of one or more of the bacteria Gemmiger, Lachnospiraceae G, Intestimonas butyriciproducens, Eggerthella lenta, Lawsonibacter, Faecalibacterium prausnitzii (1, 9, 2, 6), Oscillospirales (3, 5), Faecalibacterium, Roseburia intestinalis, Ruthenibacterium lactatiformans, Lachnoclostridium sp./Clostridium sp., Acutalibacteraceae 3, Clostridium sp., Collinsella bouchesdurhonensis, Firmicutes bacterium CAG 103/Clostridium sp., Dysomobacter welbionis, Ruminococcus sp., Clostridiales bacterium, Clostridia bacterium, in a subject having a faecal microbiome gene count similar to or lower than the faecal microbiome gene count of the positive control sample or samples or statistically relevant value obtained from the population of control samples, for example a mean value, for example wherein the faecal microbiome gene count of the sample obtained from the mammal is at least is at least 5% lower, such as at least 10% lower, 15%, 20%, 25%, 30%, 35%, 40%, 50%, 60%, 70%, 80%, 90%, at least 100% lower, such as at least 200% lower greater than the faecal microbiome gene count of the positive control sample or samples, wherein the composition comprises an extract obtained or obtainable from Aronia melanocarpa.

In a preferred embodiment, the bacteria increased are selected from the beneficial bacterial Faecalibacterium, Roseburia intestinalis and/or Eggerthella lenta

The present invention may also provide a composition for use in increasing microbiome diversity as measured by faecal microbiome gene count and/or increasing levels of one or more of the bacteria Gemmiger, Lachnospiraceae G, Intestimonas butyriciproducens,

Eggerthella lenta, Lawsonibacter, Faecalibacterium prausnitzii (1, 9, 2, 6), Oscillospirales (3, Faecalibacterium, Roseburia 5), intestinalis, Ruthenibacterium lactatiformans, Lachnoclostridium sp./Clostridium sp., Acutalibacteraceae 3, Clostridium sp., Collinsella bouchesdurhonensis, Firmicutes bacterium CAG 103/Clostridium sp., Dysomobacter welbionis, Ruminococcus sp., Clostridiales bacterium, Clostridia bacterium, in a subject having a faecal microbiome gene count of less than 400,000, optionally less than 375,000, optionally less than 350,000, 300,000, 275,000, 250,000, 225,000, 200,000, 175,000, or less than 150,000, wherein the composition comprises an extract obtained or obtainable from Aronia melanocarpa. In a preferred embodiment, the bacteria increased are selected from the beneficial bacterial Faecalibacterium, Roseburia intestinalis and/or Eggerthella lenta

The present invention may also provide a composition for use in increasing microbiome diversity as measured by faecal microbiome gene count and/or increasing levels of beneficial bacterial such as Faecalibacterium, Roseburia intestinalis and/or Eggerthella lenta in a mammal (i.e. human) having a faecal microbiome gene count of less than 400,000, optionally less than 375,000, optionally less than 350,000, 300,000, 275,000, 250,000, 225,000, 200,000, 175,000, or less than 150,000, wherein the composition comprises an extract obtained or obtainable from Aronia melanocarpa.

The present invention may also provide a method for increasing microbiome diversity as measured by faecal microbiome gene count and/or increasing levels of beneficial bacterial, such as Faecalibacterium, Roseburia intestinalis and/or Eggerthella lenta in a mammal (i.e. human) having a faecal microbiome gene count of less than 400,000, optionally less than 375,000, optionally less than 350,000, 300,000, 275,000, 250,000, 225,000, 200,000, 175,000, or less than 150,000, wherein the method comprises the administration of a composition comprising an extract obtained or obtainable from Aronia melanocarpa to a subject in need thereof.

The present invention may also provide a method for increasing microbiome diversity as measured by faecal microbiome gene count and/or increasing levels of beneficial bacterial, such as Faecalibacterium, Roseburia intestinalis and/or Eggerthella lenta in a mammal (i.e. human) having a faecal microbiome gene count similar to or lower than the faecal microbiome gene count of the positive control sample or samples or statistically relevant value obtained from the population of control samples, for example a mean value, for example wherein the faecal microbiome gene count of the sample obtained from the mammal is at least is at least 5% lower, such as at least 10% lower, 15%, 20%, 25%, 30%, 35%, 40%, 50%, 60%, 70%, 80%, 90%, at least 100% lower, such as at least 200% lower greater than the faecal microbiome gene count of the positive control sample or samples, wherein the method comprises the administration of a composition comprising an extract obtained or obtainable from Aronia melanocarpa to a subject in need thereof.

As will be appreciated by the person skilled in the art, as used herein, the term “obtainable from” means that the extract may be obtained from a plant or may be isolated from the plant, or may be obtained from an alternative source, for example by chemical synthesis or enzymatic production. Whereas the term “obtained” as used herein, means that the extract is directly derived from the plant source.

The extract obtained or obtainable from Aronia melanocarpa, may be in the form of a liquid or a solid. Typically, the extract may be in the form of a solid, i.e. a powder.

All references herein to an extract obtained from or obtainable from Aronia melanocarpa will typically refer to extracts obtained from or obtainable from the juice of Aronia melanocarpa. The juice may have been concentrated.

In some aspects, the Aronia melanocarpa juice (concentrated juice) may be extracted using water only. This extract may be referred to as the water extract.

In other aspects, the Aronia melanocarpa juice (concentrated juice) may be extracted using alcohol, such as ethanol. This extract may be referred to as the alcohol extract, such as the ethanol extract.

In a preferred aspect, the Aronia melanocarpa juice (concentrated juice) may be extracted using a mixture of alcohol and water, such as ethanol and water. This extract may be referred to as the hydro-alcoholic extract, such as the hydro-ethanolic extract.

In other aspects, the Aronia melanocarpa juice (concentrated juice) may be extracted using an organic solvent that is not an alcohol, such as acetone. This extract may be referred to as the organic extract or the acetone extract.

Additionally the polyphenols of the Aronia melanocarpa juice concentrate (or any of the extracts mentioned before) may be further purified to obtain extracts enriched in polyphenols using, for example polyphenol absorbing columns or any other technique known in the art that provides high purified polyphenols.

Typically the target polyphenols are absorbed by the resin allowing the remaining solids to pass through the column. Water and ethanol are then used to obtain an eluate which is then concentrated to provide a native extract.

Typically, the extract obtained or obtained from Aronia melanocarpa comprises 10% or more of total polyphenols (based on catechin), such as 20% or more, 30% or more, such as 40% or more by weight of the extract. In a preferred embodiment, the total polyphenols (based on catechin) is 40% or more.

The composition comprising an extract obtained or obtainable from Aronia melanocarpa, may consist or consist essentially of the extract or may comprise the extract in combination with non-toxic pharmaceutically acceptable excipients (or ingredients). These excipients (or ingredients) may, for example, be: inert diluents, such as calcium carbonate, sodium carbonate, lactose, calcium phosphate or sodium phosphate; granulating and disintegrating agents, for example, corn starch, maltodextrin or alginic acid; binding agents, for example, starch, gelatine or acacia; or lubricating agents, for example magnesium stearate, stearic acid, talc and mixtures thereof. In a preferred embodiment, the product has not carrier.

For the avoidance of doubt, preferences, options, particular features and the like indicated for a given aspect, feature or parameter of the invention should, unless the context indicates otherwise, be regarded as having been disclosed in combination with any and all other preferences, options, particular features and the like as indicated for the same or other aspects, features and parameters of the invention.

It should be noted that when we use the term “consisting essentially of” or “consists essentially of” we mean that the composition or extract or juice being described must contain the listed ingredient(s) and may also contain small (for example up to 2% by weight, or up to 1% or up to 0.1% or 0.01% by weight) of other ingredients provided that any additional ingredients do not affect the essential properties of the composition or extract. When we use the term “consisting of” we mean that the composition being described must contain the listed ingredient(s) only. These terms can be applied in an analogous manner to processes, methods and uses.

The extract obtained or obtainable from Aronia melanocarpa contains/comprises polyphenols.

Typically, in the composition comprising an extract obtained or obtained from Aronia melanocarpa, the total polyphenols (based on catechin) is 10% or more, such as 20% or more, 30% or more, such as 40% or more by weight of the composition. In a preferred embodiment, the total polyphenols (based on catechin) is 40% or more.

In any of the compositions for use and methods of the invention, the dosage of the composition for treating cardiovascular disease and/or gut dysbiosis may be from about 100 to about 1000 mg/day by weight of the composition, such as from about 400 to about 600 mg/day or about 500 mg/day by weight of the composition.

In some embodiments the dosage for treating cardiovascular disease and/or gut dysbiosis may be:
a) from about 100 to about 1000 mg/day by weight of the composition, optionally between 100 to 1000 mg/day, or between 200 and 800 mg/day, or between 400 and 600 mg/day by weight of the composition;
b) less than or equal to 1000 mg/day, optionally less or equal to than 900 mg/day, 800 mg/day, 700 mg/day, 600 mg/day, 500 mg/day, 400 mg/day, 300 mg/day, 200 mg/day or less than or equal to 100 mg/day by weight of the composition; and/or
c) greater than or equal to 100 mg/day, or optionally greater than or equal to 200 mg/day, 300 mg/day, 400 mg/day, 500 mg/day, 600 mg/day, 700 mg/day, 800 mg/day, 900 mg/day or greater than or equal to 1000 mg/day.

The dosage of the composition typically may provide from about 100 mg/day to about 500 mg/day of polyphenols by weight of the composition, such as about 200 mg/day.

In some embodiments the dosage for treating cardiovascular disease and/or gut dysbiosis provides:
a) from about 100 mg/day to about 500 mg/day of polyphenols by weight of the composition, optionally from about 200 to 400 mg/day, optionally about 300 mg/day of polyphenols by weight of the composition;
b) less than or equal to 500 mg/day of polyphenols by weight of the composition, optionally less than 400 mg/day, 300 mg/day, 200 mg/day, or less than or equal to 100 mg/day of polyphenols by weight of the composition; and/or
c) greater than or equal to 100 mg/day, optionally greater than or equal to 200 mg/day, 300 mg/day, 400 mg/day, or greater than or equal to 500 mg/day of polyphenols by weight of the composition.

The composition may be taken once a day, or more than once a day depending on the dosage required.

Typically, the composition may be taken for at least one week, for example, at least six weeks or at least 12 weeks.

In the method defined above determining the faecal microbiome gene count may comprise conducting shotgun testing on faecal samples.

Typically, in the method defined above, the method may comprise identifying a subject as likely to respond to the treatment when the gene count is 400,000 or less.

In the compositions for use and methods of the invention, treating cardiovascular disease and/or gut dysbiosis as defined above may preferably be in mammals, (i.e. humans).

As used in the specification and claims, the singular forms “a,” “an,” and “the” include plural references unless the context clearly dictates otherwise. For example, “a microbe” can include a plurality of microbes.

The term “microbiome,” “microbiota,” and “microbial habitat” can be used interchangeably herein and can refer to the ecological community of microorganisms that live on or in a subject's body. In the present invention, the microbiome may particularly relate to that found in the gastrointestinal tract.

The terms “treatment” and “treating,” as used herein, refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, a composition can be administered to a subject at risk of developing a particular disease, condition, or symptom, or to a subject reporting one or more of the physiological symptoms of a disease, even though the disease, condition, or symptom may not have yet been manifested.

As used herein, “administer,” “administering,” “administration,” and derivatives thereof refer to the methods that may be used to enable delivery of agents or compositions to the desired site of biological action. These methods include, but are not limited to, parenteral administration (e.g., intravenous, subcutaneous, intraperitoneal, intramuscular, intravascular, intrathecal, intranasal, intravitreal, infusion and local injection), transmucosal injection, oral administration, administration as a suppository, and topical administration. In the present invention the preferred route of administration may be oral administration.

The term “effective amount” or “therapeutically effective amount” refers to the quantity of a composition, for example a composition comprising microbes of the present disclosure, that is sufficient to result in a desired activity upon administration to a subject in need thereof. Within the context of the present disclosure, the term “therapeutically effective” refers to that quantity of a composition that is sufficient to delay the manifestation, arrest the progression, relieve or alleviate at least one symptom of a disorder treated by the methods of the present disclosure.

### FIGURES

FIG. 1—Study design of the clinical study. ABPM, ambulatory blood pressure monitor; FMD, flow mediated dilation; IPAQ, International physical activity questionnaire.

FIG. 2—Boxplot showing changes from baseline in gut microbiome gene count after 12-week intervention. The significance was tested by applying unpaired Wilcoxon rank-sum tests for variation at 12 weeks compared with baseline in Aronia versus Control groups. P-value represents the change from Control.

FIG. 3—Bar plot of the species significantly different between Aronia and Control groups after changes from baseline. The bar plot shows the Cliff delta effect size metric (absolute value >0.2) and increasing color intensity depicts an increasing Cliff delta value.

FIG. 4: Boxplot showing statistical differences between Aronia Responders (R), Non-Responders (NR), and Control (C) groups for changes in 24 h and awake SBPbr and 24 h and awake DBPbr following 12-week consumption. For each comparison, significance was tested by applying a Kruskal Wallis test with Bonferroni adjustment. SBPbr: brachial systolic blood pressure, DBPbr: brachial diastolic blood pressure.

FIG. 5—Boxplot showing statistical differences between Aronia Responders (R), Non-Responders (NR), and Control (C) groups for the gut microbiome gene count at baseline. The significance was tested by applying a Dunn post-hoc test after Kruskal Wallis test and Bonferroni adjustment.

FIG. 6—Cuneiform plot showing bacterial taxa significantly enriched in Aronia Responders (R), Non-Responders (NR) or Control (C) at baseline. For each comparison, a triangle pointing up indicates that species are enriched in the first mentioned group (NR for a and b and R for c). Signed effect size are shown through marker direction and colour, hue and size represent absolute effect size. Solid borders indicate significance. Markers not shown had no differences in statistical analysis.

FIG. 7—Spearman's correlation plot between baseline levels of B. adolescentis and chronic changes in 24 h DBP (Aronia: triangle; Control: circle). On the right, boxplots reporting the delta changes in 24 h DBP. On top, boxplots for log 10 baseline levels of B. adolescentis.

FIG. 8—Boxplot showing changes from baseline in gut microbiome gene count after 12-week intervention in Aronia Responders (R), Non-Responders (NR) or Control (C) groups. The significance for each comparison was tested by applying a Dunn post-hoc test after Kruskal Wallis test and Bonferroni adjustment.

FIG. 9—Species significantly different between R and NR after 12 weeks of intervention. The bar plot shows the Cliff delta value (an effect size metric) of all species significantly and relevantly (absolute value of Cliff delta higher than 0.3) contrasted between R and NR. Increasing colour intensity depicts an increasing absolute Cliff delta value.

The present invention will be further described with reference to the following, non-limiting examples:

### EXAMPLES

### Example 1—Preparation of a Composition Comprising an Extract Obtained or Obtainable from Aronia melanocarpa

Frozen Aronia melanocarpa juice concentrate is diluted with water and applied to a polyphenol absorbing resin column. The target polyphenols are absorbed by the resin allowing the remaining solids to pass through the column. Water and ethanol are then used to obtain an eluate which is then concentrated to provide a native extract. The concentration of polyphenols of the extract is of about 40%.

The native extract is then spray dried and combined with maltodextrin. The dried extract is then sieved and packaged.

### Example 2—Investigations into the Effect of Aronia melanocarpa Extract on Cardiometabolic Health

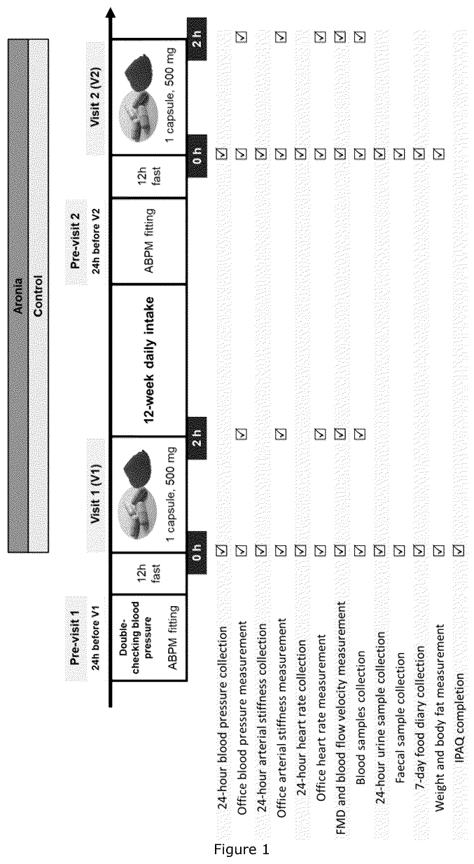

A 2-arm, double-blind, parallel, randomised, controlled trial was performed. Participants with systolic blood pressure between 120-140 and/or diastolic blood pressure between 80-90 mmHg attended 4 study visits, defined as follows: pre-visit 1, visit 1, pre-visit 2 and visit 2. A summary of the study design can be found in FIG. 1.

### Study Methodology

Pre-visits were performed 24 hours prior to visits 1 and 2. During pre-visits participants were given a bottle to collect their urine for 24 h and 24 h ambulatory BP monitors (ABPM) were fitted on the non-dominant arm with the first measurement taken.

Patients were also asked to complete 7 day food diaries prior to their visits, to avoid caffeine, alcohol, strenuous exercise and tobacco 1 hour before each visit and to fast for 12 hours before visit 1 and 2.

Measurements of peripheral office BP, FMD, PWV, and AIx, as well as blood samples, were all taken at baseline (0 h), then again 2 h post-acute consumption of the interventional product, on visit 1 and visit 2. Faecal samples were collected during both visits 1 and 2 and stored immediately at −80° C.

Participants were instructed to take 1 capsule of the composition comprising an extract obtained or obtainable from Aronia melanocarpa (500 mg) or placebo every morning with a glass of water, ideally with food. The first capsule was delivered after the first round of measurements on visit 1, marking the start of the 12 weeks of consumption. The last capsule was also taken within the research unit on the last day (visit 2), following the first set of vascular measurements.

Subjects were followed-up every month throughout the 12-week period via email to ensure good compliance and record any potential adverse event.

The primary outcome of this study was the effect of Aronia berry extract (Aronia) versus placebo (Control) on ambulatory 24-hour SBP and DBP at 12 weeks post-consumption.

Second endpoints included the effect of the extract on office BP, office and 24-hour heart rate, arterial stiffness (measured as PWV and AIx), blood lipids (total, HDL and LDL cholesterol, triglycerides), blood cortisol levels compared with placebo after 12 weeks of daily consumption, as well as the safety and tolerability of the Aronia berry extract.

Secondary objectives also included the investigation of the effect of Aronia extract versus placebo on FMD and blood flow velocity at 2 h and 12 weeks post-consumption.

Tertiary outcomes comprised the analysis of blood samples taken at all timepoints to assess Aronia PP metabolites, the analysis of 24-hour urine samples to investigate excretion of PP metabolites, as well as the analysis of the gut microbiome on faecal samples 12 weeks after consumption of the Aronia berry extract or placebo.

The trial was conducted in agreement to the guidelines stated in the current revision of the Declaration of Helsinki. All procedures were approved by King's College London Ethics Committee (RESCM-17/18-5283) and the trial was registered at ClinicalTrials.gov under the reference NCT03434574.

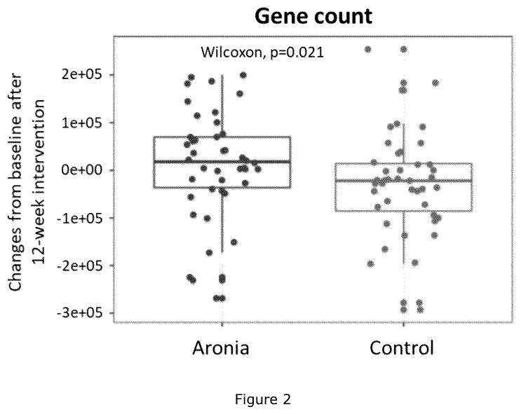

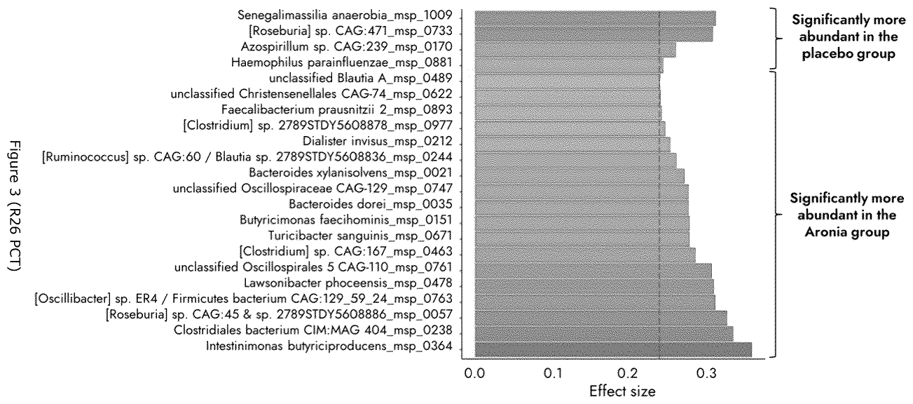

Twenty-four-hour ambulatory peripheral (brachial) and central (aortic) BP, as well as heart rate (HR), were measured using an Arteriograph24™ (TensioMed, Budapest, Hungary) (FIG. 2.1). Twenty-four-hour SBP, DBP and HR were measured every 30 minutes over the 24 hours separating the fitting of the cuff and the retrieving of the data.

Supine office peripheral (brachial) BP and heart rate were measured using an automated clinical digital sphygmomanometer OMRON M3 (OMRON Healthcare UK Ltd, Milton Keynes, UK) at the upper right arm in supine position, after 10 min of rest in a quiet room with the arm rested at heart level, legs uncrossed and supported, back supported and an empty bladder, according to the recommendations of the American Heart Association (Muntner et al. 2019).

Twenty-four-hour PWV and AIx were measured every 30 minutes over the 24 hours separating the pre-visits and the visits using Arteriograph24™. Participants were asked to fill a 24-hour activity record to indicate their daily activities day and evaluate their awake and asleep period.

Volunteers were instructed to lie down and remain silent for the duration of this measurement. PWV (expressed in m/s) was assessed via applanation tonometry using SphygmoCor® (Smart Medical, Gloucestershire, UK). Briefly, PWV is derived from the difference between transit time from the heart to femoral artery pulse and that from the heart to carotid artery pulse. The sensor of the device captures the time lapse between pulse waves at both arteries and is thus able to determine the wave velocity, as the carotid-to-heart and heart-to-femoral distances were measured beforehand with a tape measure. Results with a standard deviation of less than 8% were included in the analysis.

Ascending pressure waveforms are generated at the aorta level and involve a reflected wave which is dependent on the vessels structure and general peripheral resistance. As this latter is reduced at the body periphery, pressure is decreased from the aorta to the wrist. The waveform thus has a different shape when measured at the aorta level compared with the wrist. AIx is measured as the ratio of the central pulse pressure at aorta level and the reflected pulse pressure (called augmentation pressure). AIx is a relevant indicator of the arterial stiffness status of the vascular system of an individual.

Faecal samples were collected as close as possible of each study visit in OMNIgene GUT self-collection tubes (DNA Genotek Inc., Ottawa, Canada) and stored at −80° C. until further analysis. Participants were asked to record the collection date.

Microbiome shotgun metagenomics analysis was performed by CosmosID, Inc. (Rockville, MD, USA). Briefly, extracted DNA samples were quantified using Qubit 4 fluorometer and Qubit™ dsDNA HS Assay Kit (Thermofisher Scientific). DNA libraries were then prepared using the Nextera XT DNA Library Preparation Kit (Illumina) and Nextera Index Kit (Illumina) following the manufacturer's protocol with slight modifications. The standard protocol was used for total DNA input of 1 ng. Genomic DNA was fragmented using a proportional amount of Illumina Nextera XT fragmentation enzyme. Combinatory dual indexes were added to each sample followed by 12 cycles of PCR to construct libraries. DNA libraries were purified using AMpure magnetic Beads (Beckman Coulter) and eluted in QIAGEN EB buffer and were quantified using Qubit 4 fluorometer and Qubit™ dsDNA HS Assay Kit. DNA libraries were finally pooled together for sequencing on the Illumina HiSeqX. Raw reads from metagenomics samples were analysed by CosmosID metagenomic software (CosmosID Inc., Rockville, MD) to reveal strain level identification of microbes in the specimen as described elsewhere (Ponnusamy et al. 2016; Hasan et al. 2014; Ottesen et al. 2016). In short, the system utilizes a high-performance data mining k-mer algorithm and highly curated dynamic comparator databases that rapidly disambiguate millions of short reads into the discrete genomes or genes engendering the particular sequences. This query of reads enables the sensitive yet highly precise detection and taxonomic classification of microbial reads. The resulting statistics were analysed to return the fine-grain taxonomic and relative abundance estimates for the microbial datasets.

Bioinformatic analysis and statistical modelling of gut microbiome was then performed with the help of INRAe-Metagenopolis (Paris, France). Metagenomic Species Pangenome (MSP) are repertoires of genes composed of genes presents in all strains (“core genes”) and genes present in only some of them (“accessory genes”) (Medini, D., et al. 2005. ‘Current opinion in genetics & development, 15(6), pp. 589-594.). The clustering tool MSPminer, developed by Plaza Oñate and colleagues (Plaza Oñate, F., et al. 2019. ‘Bioinformatics, 35(9), pp. 1544-1552) can group co-abundant genes into MSP, based on the largest gene abundance table of the human gut microbiota available and containing 10.4 million genes isolated from 1267 faecal samples. MSP abundance profiles were calculated as the mean abundance of 100 markers genes, defined as the robust base of each MSP cluster.

The association of multiple omics' data was carried out through a parallel and vertical integration scheme. The variable selection was performed in per single omic level and more precisely, for each omic dataset a univariate statistical analysis (Wilcoxon test) was performed to investigate the differences of individual molecular levels between the given phenotypes of interest (Aronia versus Control). The parallel integration treats each type of omics measurements equally and the integration has been identified simultaneously in a joint model by collecting and correlating all the per-single significantly different features. Spearman correlations were calculated between concentrations of plasma and urine metabolites, clinical outcomes, and gut functions, and microbial species.

### Study Results

A total of 323 volunteers visited the unit and were screened to be part of the study, among whom 221 were excluded and 102 (47 men, 55 women) were enrolled and assigned randomly to one of the 2 intervention groups to constitute the intention-to-treat (ITT) population. Two participants discontinued the intervention for personal reasons and 3 were lost to follow-up, accounting for a total 5% drop-out. Ninety-seven participants completed all visits and were thus included in the analysis. As well as these 5 dropouts, 5 participants were removed from the per protocol (PP) population due to low compliance (<80%, n=1), history of aortic stenosis (n=1), abnormal increase in GGT, ALT and TG at visit 2 along with a high alcohol consumption at screening visit (n=1), abnormal rise of cholesterol level associated with thyroid deficiency (n=1) and suspicion of flu at visit 2 (n=1).

All clinical results are presented in the ITT population. All microbiome analyses were performed on the PP population. Moreover, several fecal samples were not analyzed due to the consumption of antibiotics within 3 months (n=4), missing faecal samples (n=2), and failure to pass the quality check based on hierarchical clustering (n=1). The remaining population considered for all microbiome analyses consisted of 85 subjects (Aronia, n=42, and Control, n=43).

No statistically significant differences between Control and Aronia group were found at baseline, except the asleep heart rate which was higher in the Aronia group (p=0.031).

The analysis of 7-day food diaries completed by participants at baseline (1-week prior the start of the study) showed no significant differences between the diet of participants in the Aronia and Control groups in terms of micro-, macronutrients and PP at baseline, apart from vitamin B3 (p=0.047) which was higher in the Control group (36.2 versus 40.8 mg).

24-hour, awake, and asleep PWV, central AIx (AIxao) and peripheral AIx (AIxbr) were measured prior both visit 1 and 2 using Arteriograph24™. Aronia extract consumption for 12 weeks led to a significant decrease in 24 h and awake peripheral and central AIx in comparison with Control (Δ24h AIxbr=−6.8%, p=0.003; Δ24h AIxao=−3.3%, p=0.006, ΔAwake AIxbr=−6.1%, p=0.020; ΔAwake AIxao=−2.9%, p=0.034). A non-significant trend for a decrease in Aronia was also observed for asleep AIxao and AIxbr. A significant reduction in awake PWV was also observed in Aronia compared to Control (APWV=−0.24 m/s, p<0.05). Similar results were found for BACO analysis. The results are shown in Table 1.

TABLE 1
Effects of aronia berry extract after 12-week consumption
on 24-hour, awake and asleep central PWV and peripheral
and central Alx in the ITT population and each intervention
group, following BAB analysis.

| Parameter | Unit | CFB Aronia | CFB Control | CFC | p-value |
|---|---|---|---|---|---|
| 24-hour PWVao | m/s | −0.05 ± 0.11 | 0.11 ± 0.11 | −0.15 | 0.336 |
| Awake PWVao | m/s | −0.13 ± 0.14 | 0.11 ± 0.14 | −0.24 | 0.049 |
| Asleep PWVao | m/s | −0.11 ± 0.13 | 0.18 ± 0.13 | −0.29 | 0.128 |
| 24-hour Alxao | % | −0.99 ± 0.81 | 2.28 ± 0.79* | −3.26 | 0.006 |
| Awake Alxao | % | −1.05 ± 1.01 | 1.85 ± 0.99 | −2.89 | 0.034 |
| Asleep Alxao | % | −0.97 ± 1.28 | 1.80 ± 1.26 | −2.76 | 0.134 |
| 24-hour Alxbr | % | −2.33 ± 1.58 | 4.45 ± 1.54* | −6.78 | 0.003 |
| Awake Alxbr | % | −2.62 ± 2.03 | 3.51 ± 2.01 | −6.13 | 0.020 |
| Asleep Alxbr | % | −1.91 ± 2.52 | 3.54 ± 2.49 | −5.45 | 0.134 |

Values expressed as mean ± SD. Alx, augmentation index; ao, aortic; br, brachial; CFB, changes from baseline; CFC, changes from Control; PWV, pulse wave velocity.
*Significance at p < 0.05 between baseline and 12 weeks.

### Discussion

A novel finding of this work is the observed significant decrease in 24 h and awake AIxao (6.8 and 6.1%, respectively) and 24 h and awake AIxbr (3.3 and 2.9%, respectively), as well as a decrease in awake PWV of 0.24 m/s following 12-week consumption of Aronia in comparison with placebo. PWV and AIx are gold standard techniques for the assessment of arterial stiffness, which is an evaluation of both arterial structure and function. Both techniques are known to be strongly correlated with CVD.

### Example 4—Investigations into the Effect of Aronia melaocarpa Extract on Gut Microbiota

The objective of this investigation was to investigate the impact of Aronia consumption on gut microbiome abundance and composition, and to explore associations between gut microbiome, Aronia polyphenol metabolites and vascular outcomes.

Faecal samples were collected at baseline and after 12 weeks of daily consumption of the Aronia extract or the placebo and immediately stored at −80° C. Detailed information regarding the collection and processing of faecal samples can be found in Example 2.

The richness of the samples was evaluated at gene and MSP levels. Significant differences were found for gene count when considering the changes from baseline between Aronia and Control groups (p=0.021) (FIG. 2).

These results indicate that consumption of Aronia extract for 12 weeks led to an increase in the gene count, suggesting a favourable increase in the gut microbiota richness after Aronia consumption.

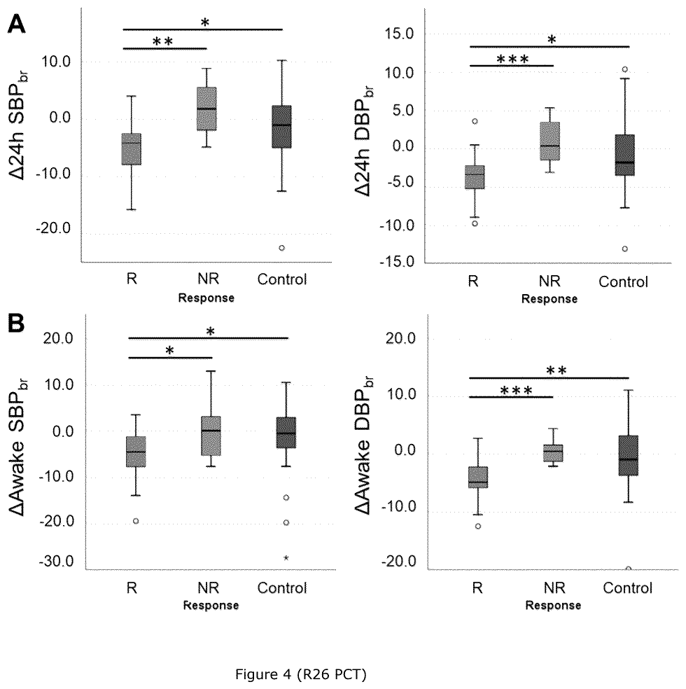

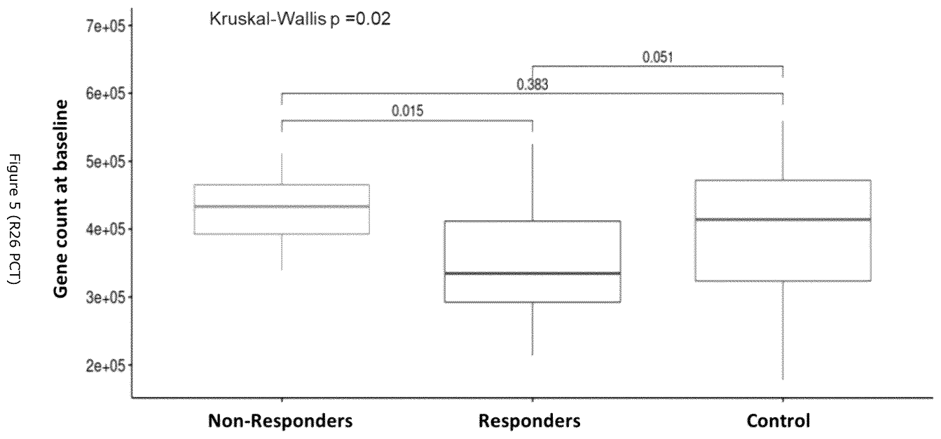

A similar analysis of the bacterial abundance and composition was conducted for the changes from baseline to depict the differences in species abundance between the 2 treatment groups following 12-week daily intake of Aronia extract or placebo (FIG. 4). A total of 18 and 4 species were significantly more abundant in Aronia and Control groups, respectively. Among them, Intestinimonas butyriciproducens was the most abundant bacteria in the Aronia group compared to Control, following the 12-week intervention. Other bacteria significantly more abundant in Aronia group were Clostridiales bacterium, Oscillibacter sp. Firmicutes bacterium CAG 103, Lawsonibacter asaccharolyticus, Oscillospirales 5, Clostridium sp., Butyricimonas faecihominis, Turicibacter sanguinis, Bacteroides dorei, Oscillospiraceae, Bacteroides xylanisolvens, Ruminococcus sp./Blautia sp., Dialister invisus, Flavonifractor sp./Clostridium sp., Faecalibacterium prausnitzii 2, Christensenellales, and Blautia A.

The beneficial effect of treatment on gut microbiome and its impact on several functional relevant pathways (|cliff delta|>0.2) was investigated (Table 2). Thirteen pathways were identified as relevant and associated with Aronia group including 7 statistically significant. Among them, γ-aminobutyric acid (GABA) production, a neurotransmitter produced by bacteria such as Lactobacillus to decrease the intracellular pH; histidine degradation and pyruvate to ferredoxin reduction, both implicated in the formation of the SCFA acetate γ-hydroxybutyrate degradation, related to the pathway resulting in the production of butyrate superoxide dismutase, an antioxidant enzyme involved in the oxidative stress response; polysaccharide A, a capsular carbohydrate found in Bacteroides fragilis and presenting anti-inflammatory properties, and pathways involved in the production of the SCFA propionate. Three pathways were related with Control group, including 2 statistically significant.

TABLE 2
Functional pathways related to Aronia and Control
groups when considering changes from baseline.

| Pathways | Group | Cliff delta | p-value |
|---|---|---|---|
| Lipopolysaccharide transport system | Aronia | 0.24 | 0.060 |
| Phosphotransferase system | Aronia | 0.28 | 0.028 |
| MtrB-MtrA (osmotic stress | Aronia | 0.25 | 0.047 |
| response) regulatory system | | | |
| Propionate synthesis II | Aronia | 0.22 | 0.080 |
| Y-Hydroxybutyrate degradation | Aronia | 0.24 | 0.053 |
| Polysaccharide A | Aronia | 0.28 | 0.022 |
| Superoxide dismutase | Aronia | 0.21 | 0.091 |
| Propionate production | Aronia | 0.22 | 0.080 |
| Pyruvate:ferredoxin oxidoreductase | Aronia | 0.27 | 0.035 |
| Histidine degradation | Aronia | 0.30 | 0.018 |
| GABA biosynthesis | Aronia | 0.22 | 0.037 |
| Siroheme biosynthesis | Aronia | 0.23 | 0.068 |
| Nitrogen fixation | Aronia | 0.26 | 0.028 |
| Osmoprotectant transport system | Control | −0.27 | 0.035 |
| LiaS-LiaR (cell wall stress | Control | −0.24 | 0.058 |
| response) regulatory system | | | |
| Pyrimidine deoxyribonucleotide | Control | −0.24 | 0.042 |
| biosynthesis | | | |

### Discussion

It's well established that host health is influenced by gut microbiome composition as well as the role of diet on microbiome-mediated outcomes. Thus, decreased gene richness of the intestinal microbial ecology has been already reported in gut dysbiosis condition associated with disease (Le Chatelier, et al. Nature, 500 (7464), pp. 541-546.) as well as with prehypertensive and hypertensive status both in rats and human studies (Li et al. 2017). Our analysis showed the beneficial impact exerted by Aronia extract consumption over gut microbiome composition as observed for the increased levels of gene count in the Aronia compared to Control group, possibly contributing to the positive improvements in observed arterial outcomes.

Moreover, several beneficial species were found to be enriched in the Aronia compared with Control group following 12-weeks' supplementation. Noteworthy, increased levels of butyrate-producers taxa species were reported like Faecalibacterium prausnitzii (Miquel, S., et al. Gut microbes, 5(2), pp. 146-151), Lawsonibacter asaccharolyticus (Sakamoto, M., et al. International journal of systematic and evolutionary microbiology, 68(6), pp. 2074-2081.) and Intestinimonas butyriciproducens microorganisms associated with a healthy gut layout along with B. xylanisolvens, a xylan-degrading bacterium. Xylan as dietary fibers is fermented by the human gut microbiota leading to the production of short chain fatty acids. Coherently, those observations were substantiated by the increase of potential functional modules leading to the production of propionate, a diet-related gut microbial metabolite shown to play an important role in cardiometabolic health and hypertension. (Muralitharan, R. R. et al. Journal of human hypertension, 35(2), pp. 162-169.).

### Example 5—Identification of a “Responder” Subpopulation

A high variability in the vascular response to Aronia consumption was found among the study population. We hypothesised that this interindividual variability in response could be related to differences in the gut microbiome at baseline. Responders (R) versus Non-Responders (NR) were categorized among participants from the Aronia group (n=42) based on an unsupervised method of clustering k-means.

The following variables were included in the model as relevant clinical parameters: ambulatory blood pressure (BP) (primary outcome), office SBP, ambulatory AIx and PWV, FMD and cortisol levels. All clinical parameters were primary and secondary outcomes of the RCT and included ambulatory blood pressure (BP) (primary outcome), office SBP, ambulatory AIx and PWV, FMD and cortisol levels (Table 3). The cut-off decision for each parameter was made based on clinical relevance, overall magnitude of effects and range of responses in our study, as well as the ability to perform statistical analysis on big enough groups (Table 3).

TABLE 3
Summary of the parameters included in the cluster
analysis, with detail of their cut-off limit.

| | Primary/Secondary | | |
|---|---|---|---|
| | outcome | Parameters | Cut-off |
| Ambulatory BP | Primary | Δ 24 h SBP_{br} | ≤−2 vs >−2 mmHg |
| | | Δ 24 h DBP_{br} | |
| Office BP | Secondary | Δ SBP_{br} | ≤−2 vs >−2 mmHg |
| Ambulatory Alx | Secondary | Δ 24 h Alxₐₒ | <0 vs ≥0% |
| | | Δ 24 h Alx_{br} | |
| | | Δ Awake Alxₐₒ | |
| | | Δ Awake Alx_{br} | |
| Ambulatory PWV | Secondary | Δ Awake PWV | <0 vs ≥0 m/s |
| FMD | Secondary | Δ FMD | <0.7 vs ≥0.7% |
| Cortisol levels | Secondary | Δ Cortisol | <0 vs ≥0 mmol/L |

ao, aortic;
br, brachial;
BP, blood pressure;
CVD, cardiovascular disease;
DBP, diastolic BP;
FMD, flow-mediated dilation;
PWV, pulse wave velocity;
SBP, systolic BP

The best number of clusters (k=2) was defined using the NbClust( ) function (NbClust R package) on a matrix containing the changes of the given variables. This analysis was repeated 200 times to ensure the robustness of the observations, and 37 out of the 42 volunteers were included in 2 clusters. The other 5 participants were classified as “jumping subjects” as they oscillated from one cluster to another during the repetitions.

The Aronia cluster 1 consisted of 23 volunteers and the Aronia cluster 2 consisted of 14 volunteers.

Differences among clinical parameters were investigated between the two clusters to assess the impact of the Aronia treatment in the newly defined sub cohorts. The Aronia cluster 1 had a significant reduction in the primary outcome, 24 h SBPbr of −6.6 and −3.6 mmHg compared with the Aronia cluster 2, and the Control group, respectively (p<0.01). Similar observations were found regarding 24 h DBPbr (−4.6 and −2.8 mmHg decrease in the cluster 1 compared with cluster 2 and Control, respectively, p<0.01). These observations suggest that subjects in the Aronia cluster 1 responded to Aronia extract consumption (they were “Responders”, R), while those in the Aronia cluster 2 were non-responders (NR) (FIG. 4). Moreover, significant reductions in the R group in Awake SBPbr and DBPbr were found when compared with NR and control groups (FIG. 4; −5.3 and −3.8 mmHg for awake SBPbr and −4.6 and −3.3 mmHg for awake DBPbr compared with NR and Control, respectively).

As shown in above, consumption of Aronia extract for 12 weeks led to an increase in the gene count, implicating a favourable increase in the richness of the gut microbiota after Aronia consumption.

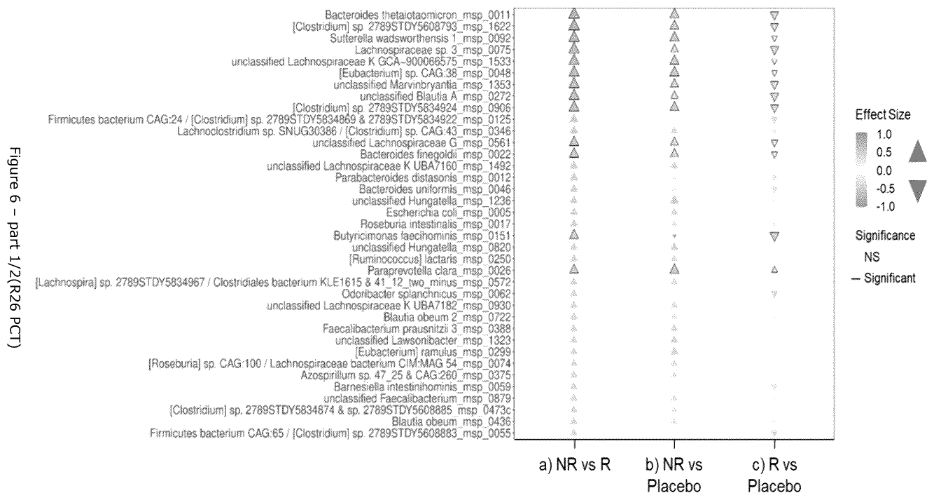

Responders had significant lower gene count at baseline compared to both NR individuals and placebo (FIG. 5).

The same conclusion was observed for species richness, although not significant after Bonferroni adjustment for multiple comparisons (Kruskal-Wallis p-value=0.18; Dunn post-hoc test p-value (R vs NR)=0.03, without correction). In addition, the gut microbiome functional analysis highlighted that subjects belonging to R group had significantly lower functional modules at baseline compared to NR group (Pearson's Chi-squared test p-value<2.2e-16), further confirming that R individuals were less rich in terms of gut bacteria composition and related potential functions compared to NR at baseline level.

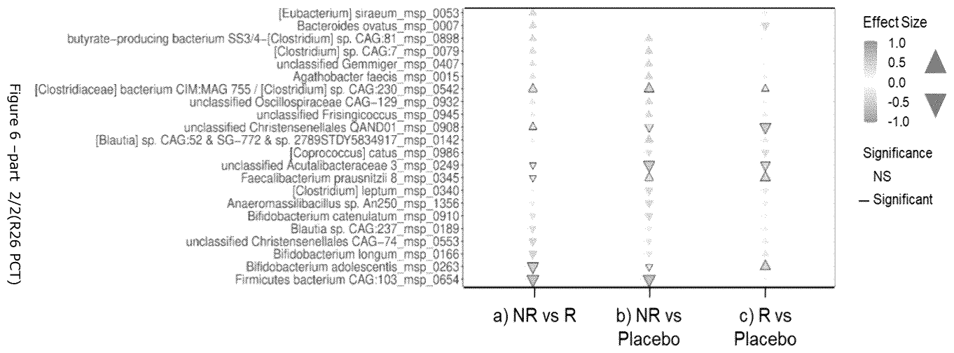

Baseline gut microbiome composition was investigated for both R and NR subgroups of the Aronia subjects. Following a Kruskal Wallis test with Dunn post-hoc test, the abundances of Faecalibacterium prausnitzii 8, Acutalibacteraceae 3, Firmicutes bacterium CAG 103 and Bifidobacterium adolescentis taxa appeared significantly enriched the R group compared to NR at baseline. Bifidobacterium adolescentis was also enriched in R compared with Control group (FIG. 6).

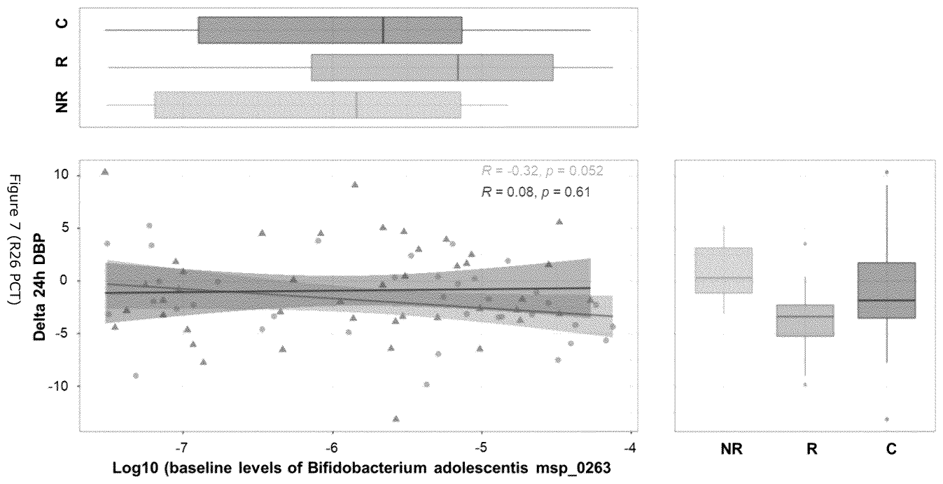

In order to predict the response to Aronia extract supplementation according to the variation in gut microbiota composition at baseline, an analysis focusing exclusively on the 4 species enriched in the R cluster (listed above) was performed. We observed that baseline abundances in Bifidobacterium adolescentis showed a significant and negative correlation with the increase in 24 h DBP for the Aronia group (Spearman's p=−0.32, p=0.05) (FIG. 7). The analysis of gut microbial composition based on the variation in response to Aronia extract treatment (Responders versus Non-Responders) revealed that the consumption of the Aronia extract can reduce BP (ambulatory 24 h and awake SBPbr and DBPbr) in subjects with low gene count at baseline compared to Control group. Among the 4 taxa significantly enriched at baseline in the R group compared with NR, a higher abundance in Bifidobacterium adolescentis was related with a greater decrease in 24 h DBP.

The impact of the Aronia extract supplementation in R and NR subgroups was assessed by comparing the gut microbiome gene count and composition of both subgroups following the 12-week intervention.

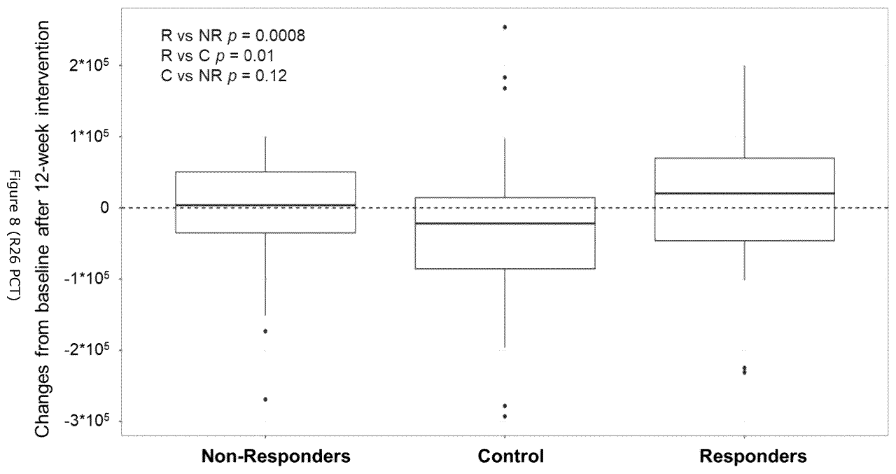

The consumption of Aronia extract for 12 weeks led to an increased gene count in the R group compared to both NR group (p=0.0008) and Control group (p=0.01) (FIG. 8). This result indicates that the beneficial effect of Aronia on this parameter was stronger individuals harbouring a lower gene count at baseline.

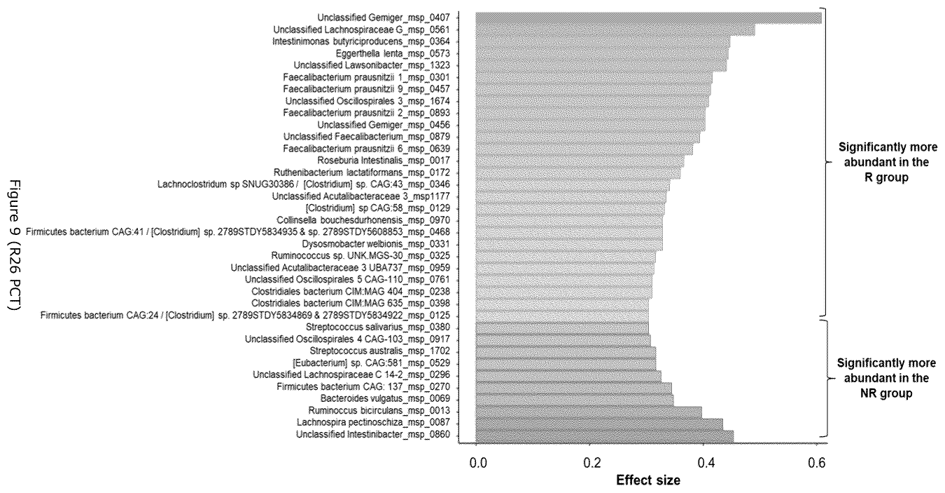

Regarding gut microbiome composition, a total of 26 and 10 species were significantly more abundant following the intervention in the R and NR groups, respectively (FIG. 9). Among the species enriched in the R group were found Gemmiger, Lachnospiraceae G, Intestimonas butyriciproducens, Eggerthella lenta, Lawsonibacter, Faecalibacterium prausnitzii (1, 9, 2, 6), Oscillospirales (3, 5), Faecalibacterium, Roseburia intestinalis, Ruthenibacterium lactatiformans, Lachnoclostridium sp./Clostridium sp., Acutalibacteraceae 3, Clostridium sp., Collinsella bouchesdurhonensis, Firmicutes bacterium CAG 103/Clostridium sp., Dysomobacter welbionis, Ruminococcus sp., Clostridiales bacterium, and Clostridia bacterium. On the contrary, the species significantly enriched in the NR individuals were Streptococcus salivarius, Oscillospirales 4, Streptococcus australis, Eubacterium sp., unclassified Lachnospiraceae C, Firmicutes bacterium CAG 103, Bacteroides vulgatus, Ruminococcus bicirculans, Lachnospira pectinoschiza, and Intestinibacter.

Several species enriched in the R group are known to be members of a healthy gut microbiome such as several species of the clade Faecalibacterium along with Roseburia intestinalis, a fibre-degrading gut symbiont able to impact atherosclerosis in vivo models (La Rosa et al., 2019) and Eggerthella lenta species, a human colon taxa producing the urolithin metabolites from the metabolism of pomegranate ellagitannins, recently shown to improve intestinal barrier function in a preclinical study and to be associated with lower cardiometabolic risk (Selma et al., 2018).

1. (canceled)

2. A method for treating gut dysbiosis and/or cardiovascular disease in a subject, wherein the method comprises administering a composition comprising an extract obtained from Aronia melanocarpa to a subject in need thereof.

3. The method as defined in claim 2, wherein treating cardiovascular diseases further comprises at least one of:
(a) Treating hypertension;
(b) Treating pre-hypertension;
(b) Reducing blood pressure; or
(c) Reducing arterial stiffness.

4. The method as defined in claim 2, wherein treating gut dysbiosis comprises at least one of:
(a) Increasing microbiome diversity as measured by faecal microbiome gene count; or
(b) Increasing levels of beneficial bacterial, such as Faecalibacterium prausnitzii 2, Lawsonibacter asaccharolyticus, Intestinimonas butyriciproducens, Faecalibacterium, Roseburia intestinalis and/or Eggerthella lenta.

5. The method according to claim 2, wherein the extract obtained from Aronia melanocarpa is an aqueous or hydroalcoholic extract, optionally, wherein the composition comprises at least 10%, or at least 20%, or at least 30%, or at least 40%, or at least 50% w/w polyphenols by weight of the composition.

6. (canceled)

7. (canceled)

8. The method according to claim 2, wherein the treating gut dysbiosis is in a mammal having a faecal microbiome gene count of less than 400,000, optionally less than 375,000, or optionally less than 350,000, or optionally less than 300,000, or optionally less than 275,000, or optionally less than 250,000, or optionally less than 225,000, or optionally less than 200,000, or optionally less than 175,000, or optionally less than 150,000.

9. The method according to claim 2, wherein the treating cardiovascular disease is by reducing blood pressure, optionally by reducing blood pressure in a mammal having a faecal microbiome gene count of less than 400,000, optionally less than 375,000, optionally less than 350,000, optionally less than 300,000, optionally less than 275,000, optionally less than 250,000, optionally less than 225,000, optionally less than 200,000, optionally less than 175,000, or optionally less than 150,000.

10. The method according to claim 2, wherein the subject is prehypertensive, optionally, wherein the subject has:
a systolic blood pressure of at least 120 mmHg, at least 130 mmHg, or at least 140 mmHg, or between 120 mmHg and 130 mmHg, or between 120 mmHg and 140 mmHg, and/or
a diastolic blood pressure of at least 80 mmHg, at least 90 mmHg, or between 80 mmHg and 90 mmHg.

11. (canceled)

12. The method according to claim 2, wherein the dosage for treating gut dysbiosis provides:
a) from about 100 mg/day to about 500 mg/day of polyphenols by weight of the composition, optionally from about 200 to 400 mg/day, optionally about 300 mg/day of polyphenols by weight of the composition;
b) less than or equal to 500 mg/day of polyphenols by weight of the composition, optionally less than 400 mg/day, 300 mg/day, 200 mg/day, or less than or equal to 100 mg/day of polyphenols by weight of the composition; and/or
c) greater than or equal to 100 mg/day, optionally greater than or equal to 200 mg/day, 300 mg/day, 400 mg/day, or greater than or equal to 500 mg/day of polyphenols by weight of the composition.

13. The method according to claim 2, wherein determining the abundance of the bacteria in a sample of faeces obtained from the subject is performed using shotgun sequencing.

14. A method for determining whether a subject is likely to respond to treatment with a composition that comprises an extract obtained or obtainable from Aronia melanocarpa, wherein the method comprises:
a) determining the faecal microbiome gene count in a sample of faeces obtained from the subject; and/or
b) determining the abundance of any 1, 2, 3 or all of:
i) Faecalibacterium prausnitzii 8 in a sample of faeces obtained from the subject;
ii) Acutalibacteraceae 3 in a sample of faeces obtained from the subject;
iii) Firmicutes bacterium CAG 103 in a sample of faeces obtained from the subject; and/or
iv) Bifidobacterium adolescentis in a sample of faeces obtained from the subject, optionally the method further comprises:
c) comparing the faecal microbiome gene count in the sample of faeces obtained from the subject with the faecal microbiome gene count in at least a first control sample or population of control samples; and/or
d) comparing the abundance of:
i) Faecalibacterium prausnitzii 8 in a sample of faeces obtained from the subject with the abundance of Faecalibacterium prausnitzii 8 in at least a first control sample or population of control samples;
ii) Acutalibacteraceae 3 in a sample of faeces obtained from the subject with the abundance of Acutalibacteraceae 3 in at least a first control sample or population of control samples;
iii) Firmicutes bacterium CAG 103 in a sample of faeces obtained from the subject with the abundance of Firmicutes bacterium CAG 103 in at least a first control sample or population of control samples; and/or
iv) Bifidobacterium adolescentis in a sample of faeces obtained from the subject with the abundance of Bifidobacterium adolescentis in at least a first control sample or population of control samples, optionally
wherein the at least a first control sample or population of control samples is a negative control sample or population or control samples.

15. (canceled)

16. (canceled)

17. The method of claim 14, wherein the negative control sample or population of control samples are faecal samples taken from one or more than one negative control subjects that have been determined to not respond to treatment with a composition that comprises an extract obtained or obtainable from Aronia melanocarpa,
wherein the samples were taken from the one or more than one control subjects prior to treatment with a composition that comprises an extract obtained or obtainable from Aronia melanocarpa.

18. The method of claim 14, wherein the at least a first control sample or population of control samples is a positive control sample or population or control samples.

19. (canceled)

20. The method of claim 14, wherein the faecal microbiome gene count of the sample obtained from the subject is compared to:
a) the faecal microbiome gene count in a negative control sample or population or control samples, optionally wherein the negative control sample or population of control samples are faecal samples taken from one or more than one negative control subjects that have been shown to not respond to treatment with a composition that comprises an extract obtained or obtainable from Aronia melanocarpa, wherein the samples were taken from the one or more than one negative control subjects prior to treatment with a composition that comprises an extract obtained or obtainable from Aronia melanocarpa; and/or
b) the faecal microbiome gene count in a positive control sample or population or control samples, optionally wherein the positive control sample or population of control samples are faecal samples taken from one or more than one positive control subjects that have been shown to respond to treatment with a composition that comprises an extract obtained or obtainable from Aronia melanocarpa, wherein the samples were taken from the one or more than one positive control subjects prior to treatment with a composition that comprises an extract obtained or obtainable from Aronia melanocarpa.

21. The method according to claim 20 wherein:
a) where the faecal microbiome gene count of the sample obtained from the subject is substantially similar to or higher than the faecal microbiome gene count of the negative control sample or samples, the subject is considered unlikely to respond to treatment with a composition that comprises an extract obtained or obtainable from Aronia melanocarpa,
optionally wherein where the faecal microbiome gene count of the sample obtained from the subject is selected from a faecal microbiome gene count that is at least 5% greater, at least 10% greater, at least 15% greater, at least 20% greater, at least 25% greater, at least 30% greater, at least 35% greater, at least 40% greater, at least 50% greater, at least 60% greater, at least 70% greater, at least 80% greater, at least 90% greater, at least 100% greater, or such as at least 200% greater than the faecal microbiome gene count of the negative control sample or samples, the subject is considered to be unlikely to respond to treatment with a composition that comprises an extract obtained or obtainable from Aronia melanocarpa; and/or
b) where the faecal microbiome gene count in the test sample is substantially similar to or lower than the faecal microbiome gene count of the positive control sample or samples, the subject is considered likely to respond to treatment with a composition that comprises an extract obtained or obtainable from Aronia melanocarpa,
optionally wherein where the faecal microbiome gene count of the sample obtained from the subject is selected from a faecal microbiome gene count that is at least 5% lower, at least 10% lower, at least 15% lower, at least 20% lower, at least 25% lower, at least 30% lower, at least 35% lower, at least 40% lower, at least 50% lower, at least 60% lower, at least 70% lower, at least 80% lower, at least 90% lower, at least 100% lower, or at least 200% lower than the faecal microbiome gene count of the positive control sample or samples, the subject is considered to be likely to respond to treatment with a composition that comprises an extract obtained or obtainable from Aronia melanocarpa.

22. The method of claim 14 wherein:
a) the abundance of:
i) Faecalibacterium prausnitzii 8 in a sample of faeces obtained from the subject is compared to the abundance of Faecalibacterium prausnitzii 8 in at least a first control sample or population of control samples;
ii) Acutalibacteraceae 3 in a sample of faeces obtained from the subject is compared to the abundance of Acutalibacteraceae 3 in at least a first control sample or population of control samples;
iii) Firmicutes bacterium CAG 103 in a sample of faeces obtained from the subject is compared to the abundance of Firmicutes bacterium CAG 103 in at least a first control sample or population of control samples; and/or
iv) Bifidobacterium adolescentis in a sample of faeces obtained from the subject is compared to the abundance of Bifidobacterium adolescentis in at least a first control sample or population of control samples
and wherein the control sample or population of control samples are a negative control sample or population or control samples
wherein the negative control sample or population of control samples are faecal samples taken from one or more than one negative control subjects that have been shown to not respond to treatment with a composition that comprises an extract obtained or obtainable from Aronia melanocarpa, wherein the samples were taken from the one or more than one positive control subjects prior to treatment with a composition that comprises an extract obtained or obtainable from Aronia melanocarpa; and/or
b) the abundance of:
i) Faecalibacterium prausnitzii 8 in a sample of faeces obtained from the subject is compared to the abundance of Faecalibacterium prausnitzii 8 in at least a first control sample or population of control samples;
ii) Acutalibacteraceae 3 in a sample of faeces obtained from the subject is compared to the abundance of Acutalibacteraceae 3 in at least a first control sample or population of control samples;
iii) Firmicutes bacterium CAG 103 in a sample of faeces obtained from the subject is compared to the abundance of Firmicutes bacterium CAG 103 in at least a first control sample or population of control samples; and/or
iv) Bifidobacterium adolescentis in a sample of faeces obtained from the subject is compared to the abundance of Bifidobacterium adolescentis in at least a first control sample or population of control samples
and wherein the control sample or population of control samples are a positive control sample or population or control samples,
wherein the positive control sample or population of control samples are faecal samples taken from one or more than one positive control subjects that have been shown to respond to treatment with a composition that comprises an extract obtained or obtainable from Aronia melanocarpa, wherein the samples were taken from the one or more than one positive control subjects prior to treatment with a composition that comprises an extract obtained or obtainable from Aronia melanocarpa.

23. The method according to claim 14 wherein:
a) where the abundance of Faecalibacterium prausnitzii 8 in a sample of faeces obtained from the subject is substantially similar to or higher than the abundance of Faecalibacterium prausnitzii 8 in a positive control sample or samples, the subject is considered likely to respond to treatment with a composition that comprises an extract obtained or obtainable from Aronia melanocarpa, optionally wherein where the abundance of Faecalibacterium prausnitzii 8 in the sample obtained from the subject is selected from at least 5% greater, at least 10% greater, at least 15% greater, at least 20% greater, at least 25% greater, at least 30% greater, at least 35% greater, at least 40% greater, at least 50% greater, at least 60% greater, at least 70% greater, at least 80% greater, at least 90% greater, at least 100% greater, or at least 200% greater than the abundance of Faecalibacterium prausnitzii 8 in a positive control sample or samples, the subject is considered likely to respond to treatment with a composition that comprises an extract obtained or obtainable from Aronia melanocarpa;
b) where the abundance of Acutalibacteraceae 3 in a sample of faeces obtained from the subject is substantially similar to or higher than the abundance of Acutalibacteraceae 3 in a positive control sample or samples, the subject is considered likely to respond to treatment with a composition that comprises an extract obtained or obtainable from Aronia melanocarpa, optionally wherein where the abundance of Acutalibacteraceae 3 in the sample obtained from the subject is selected from at least 5% greater, at least 10% greater, at least 15% greater, at least 20% greater, at least 25% greater, at least 30% greater, at least 35% greater, at least 40% greater, at least 50% greater, at least 60% greater, at least 70% greater, at least 80% greater, at least 90% greater, at least 100% greater, or at least 200% greater than the abundance of Acutalibacteraceae 3 in a positive control sample or samples, the subject is considered likely to respond to treatment with a composition that comprises an extract obtained or obtainable from Aronia melanocarpa;
c) where the abundance of Firmicutes bacterium CAG 103 in a sample of faeces obtained from the subject is substantially similar to or higher than the abundance of Firmicutes bacterium CAG 103 in a positive control sample or samples, the subject is considered likely to respond to treatment with a composition that comprises an extract obtained or obtainable from Aronia melanocarpa, optionally wherein where the abundance of Firmicutes bacterium CAG 103 in the sample obtained from the subject is selected from at least 5% greater, at least 10% greater, at least 15% greater, at least 20% greater, at least 25% greater, at least 30% greater, at least 35% greater, at least 40% greater, at least 50% greater, at least 60% greater, at least 70% greater, at least 80% greater, at least 90% greater, at least 100% greater, or at least 200% greater than the abundance of Firmicutes bacterium CAG 103 in a positive control sample or samples, the subject is considered likely to respond to treatment with a composition that comprises an extract obtained or obtainable from Aronia melanocarpa;
and/or
d) where the abundance of Bifidobacterium adolescentis in a sample of faeces obtained from the subject is substantially similar to or higher than the abundance of Bifidobacterium adolescentis in a positive control sample or samples, the subject is considered likely to respond to treatment with a composition that comprises an extract obtained or obtainable from Aronia melanocarpa, optionally wherein where the abundance of Bifidobacterium adolescentis in the sample obtained from the subject is selected from at least 5% greater, at least 10% greater, at least 15% greater, at least 20% greater, at least 25% greater, at least 30% greater, at least 35% greater, at least 40% greater, at least 50% greater, at least 60% greater, at least 70% greater, at least 80% greater, at least 90% greater, at least 100% greater, or at least 200% greater than the abundance of Bifidobacterium adolescentis in a positive control sample or samples, the subject is considered likely to respond to treatment with a composition that comprises an extract obtained or obtainable from Aronia melanocarpa;
e) where the abundance of Faecalibacterium prausnitzii 8 in a sample of faeces obtained from the subject is substantially similar to or lower than the abundance of Faecalibacterium prausnitzii 8 in a negative control sample or samples, the subject is considered unlikely to respond to treatment with a composition that comprises an extract obtained or obtainable from Aronia melanocarpa, optionally wherein where the abundance of Faecalibacterium prausnitzii 8 in the sample obtained from the subject is selected from at least 5% lower, at least 10% greater, at least 15% greater, at least 20% greater, at least 25% greater, at least 30% greater, at least 35% greater, at least 40% greater, at least 50% greater, at least 60% greater, at least 70% greater, at least 80% greater, at least 90% greater, at least 100% lower, or at least 200% lower than the abundance of Faecalibacterium prausnitzii 8 in a negative control sample or samples, the subject is considered unlikely to respond to treatment with a composition that comprises an extract obtained or obtainable from Aronia melanocarpa;
f) where the abundance of Acutalibacteraceae 3 in a sample of faeces obtained from the subject is substantially similar to or lower than the abundance of Acutalibacteraceae 3 in a negative control sample or samples, the subject is considered unlikely to respond to treatment with a composition that comprises an extract obtained or obtainable from Aronia melanocarpa, optionally wherein where the abundance of Acutalibacteraceae 3 in the sample obtained from the subject is at least 5% lower, at least 10% lower, at least 15% lower, at least 20% lower, at least 25% lower, at least 30% lower, at least 35% lower, at least 40% lower, at least 50% lower, at least 60% lower, at least 70% lower, at least 80% lower, at least 90% lower, at least 100% lower, or at least 200% lower than the abundance of Acutalibacteraceae 3 in a negative control sample or samples, the subject is considered unlikely to respond to treatment with a composition that comprises an extract obtained or obtainable from Aronia melanocarpa;
g) where the abundance of Firmicutes bacterium CAG 103 in a sample of faeces obtained from the subject is substantially similar to or lower than the abundance of Firmicutes bacterium CAG 103 in a negative control sample or samples, the subject is considered unlikely to respond to treatment with a composition that comprises an extract obtained or obtainable from Aronia melanocarpa, optionally wherein where the abundance of Firmicutes bacterium CAG 103 in the sample obtained from the subject is selected from at least 5% lower, at least 10% lower, at least 15% lower, at least 20% lower, at least 25% lower, at least 30% lower, at least 35% lower, at least 40% lower, at least 50% lower, at least 60% lower, at least 70% lower, at least 80% lower, at least 90% lower, at least 100% lower, or at least 200% lower than the abundance of Firmicutes bacterium CAG 103 in a negative control sample or samples, the subject is considered unlikely to respond to treatment with a composition that comprises an extract obtained or obtainable from Aronia melanocarpa;
and/or
h) where the abundance of Bifidobacterium adolescentis in a sample of faeces obtained from the subject is substantially similar to or lower than the abundance of Bifidobacterium adolescentis in in a negative control sample or samples, the subject is considered unlikely to respond to treatment with a composition that comprises an extract obtained or obtainable from Aronia melanocarpa, optionally wherein where the abundance of Bifidobacterium adolescentis in the sample obtained from the subject is selected from at least 5% lower, at least 10% lower, at least 15% lower, at least 20% lower, at least 25% lower, at least 30% lower, at least 35% lower, at least 40% lower, at least 50% lower, at least 60% lower, at least 70% lower, at least 80% lower, at least 90% lower, at least 100% lower, or at least 200% lower than the abundance of Bifidobacterium adolescentis in a negative control sample or samples, the subject is considered unlikely to respond to treatment with a composition that comprises an extract obtained or obtainable from Aronia melanocarpa.

24. The method according to claim 14 wherein:
i) the abundance of Faecalibacterium prausnitzii 8 in a sample of faeces obtained from the subject is compared to the abundance of Faecalibacterium prausnitzii 8 in at least a first control sample or population of control samples;
ii) the abundance of Acutalibacteraceae 3 in a sample of faeces obtained from the subject is compared to the abundance of Acutalibacteraceae 3 in at least a first control sample or population of control samples;
iii) the abundance of Firmicutes bacterium CAG 103 in a sample of faeces obtained from the subject is compared to the abundance of Firmicutes bacterium CAG 103 in at least a first control sample or population of control samples; and
iv) the abundance of Bifidobacterium adolescentis in a sample of faeces obtained from the subject is compared to the abundance of Bifidobacterium adolescentis in at least a first control sample or population of control samples.

25. The method according to claim 14 wherein the abundance of Bifidobacterium adolescentis in the faecal sample obtained from the subject is compared to the abundance of Bifidobacterium adolescentis in:
one or more negative control samples; and/or
one or more positive control samples.

26. (canceled)

27. (canceled)

28. (canceled)

29. The method according to claim 14 wherein a response to treatment is indicated by a reduction in blood pressure, optionally wherein the method is a method for determining whether a composition that comprises an extract obtained or obtainable from Aronia melanocarpa is likely to reduce the blood pressure of a particular subject.

30. The method according to claim 14 wherein the subject is prehypertensive, optionally wherein the subject has:
a systolic blood pressure selected from of at least 120 mmHg, at least 130, at least 140 mmHg, between 120 mmHg and 130 mmHg, or between 120 mmHg and 140 mmHg, and/or
a diastolic blood pressure selected from of at least 80 mmHg, at least 90, mmHg, between 80 mmHg and 90 mmHg.

31. A composition for use in treating and/or preventing cardiovascular disease and/or gut dysbiosis in a subject, wherein the composition comprises an extract obtained or obtainable from Aronia melanocarpa and wherein the subject has been determined to be likely to respond to treatment with the composition according to claim 14.

32. (canceled)
